(19) United States
(12) Patent Application Publication
(10) Pub. No.: US 2003/0152580 A1
(43) Pub. Date: Aug. 14, 2003

(54) HLA BINDING PEPTIDES AND THEIR USES

(76) Inventors: ALESSANDRO SETTE, LA JOLLA, CA (US); JOHN SIDNEY, LA JOLLA, CA (US)

Correspondence Address: Eric K. Steffe, Esq, Sterne, Kessler, Goldstein & Fox, 1100 New York Ave, N.W., Washington, DC, 20005-3934, US

(21) Appl. No.: 08/344,824

(22) Filed: Nov. 23, 1994

Related U.S. Application Data
(63) Continuation-in-part of application No. 08/278,634, filed on Jul. 21, 1994, now abandoned.

(51) Int. Cl. A61K 39/00; A61K 39/385; C07K 1/00; C07K 14/00; C07K 17/00

(52) U.S. Cl. 424/185.1; 424/193.1; 530/350; 530/395; 530/868

(57) ABSTRACT

The present invention provides peptide compositions capable of binding glycoproteins encoded by HLA, HLA-B, and HLA-C alleles and inducing T cell activation in T cells restricted by the HLA allele. The peptides are useful to elicit an immune response against a desired antigen.

### CROSS REFERENCE TO RELATED APPLICATION

This application continuation-in-part of application U.S. Ser. No. 08/271,634 filed Jul. 21, 1994, which is incorporated herein by reference.

### BACKGROUND OF THE INVENTION

The present invention relates to compositions and methods for preventing, treating or diagnosing a number of pathological states such as viral diseases and cancers. In particular, it provides novel peptides capable of binding selected major histocompatibility complex (MHC) molecules and inducing an immune response.

MHC molecules are classified as either Class I or Class II molecules. Class II MHC molecules are expressed primarily on cells involved in initiating and sustaining immune responses, such as T lymphocytes, B lymphocytes, macrophages, etc. Class II MHC molecules are recognized by helper T lymphocytes and induce proliferation of helper T lymphocytes and amplification of the immune response to the particular immunogenic peptide that is displayed. Class I MHC molecules are expressed on almost all nucleated cells and are recognized by cytotoxic T lymphocytes (CTLs), which then destroy the antigen-bearing cells. CTLs are particularly important in tumor rejection and in fighting viral infections. The CTL recognizes the antigen in the form of a peptide fragment bound to the MHC class I molecules rather than the intact foreign antigen itself. The antigen must normally be endogenously synthesized by the cell, and a portion of the protein antigen is degraded into small peptide fragments in the cytoplasm. Some of these small peptides translocate into a pre-Golgi compartment and interact with class I heavy chains to facilitate proper folding and association with the subunit β₂ microglobulin. The peptide-MHC class I complex is then routed to the cell surface for expression and potential recognition by specific CTLs.

The MHC class I antigens are encoded by the HLA-A, B, and C loci. HLA-A and HLA-B antigens are expressed at the cell surface at approximately equal densities, whereas the expression of HLA-C is significantly lower (perhaps as much as 10-fold lower). Each of these loci have a number of alleles.

Specific motifs for several of the major HLA-A alleles (copending U.S. patent application Ser. Nos. 08/159,339 and 08/205,713, referred to here as the copending applications) and HLA-B alleles have been described. Several authors (Melief, Eur. J. Immunol., 21:2963-2970 (1991); Bevan, et al., Nature 353:852-955 (1991)) have provided preliminary evidence that class I binding motifs can be applied to the identification of potential immunogenic peptides in animal models. Strategies for identification of peptides or peptide regions capable of interacting with multiple MHC alleles has been described in the literature.

Because human population groups, including racial and ethnic groups, have distinct patterns of distribution of HLA alleles it will be of value to identify motifs that describe peptides capable of binding more than one HLA allele, so as to achieve sufficient coverage of all population groups. The present invention addresses these and other needs.

### SUMMARY OF THE INVENTION

The present invention provides compositions comprising immunogenic peptides having binding motifs for HLA alleles. The immunogenic peptides are about 9 to 10 residues in length and comprise conserved residues at certain positions such as a proline at position 2 and an aromatic residue (e.g., Y, W, F) or hydrophobic residue (e.g., L,I,V,M, or A) at the carboxy terminus. In particular, an advantage of the peptides of the invention is their ability to bind to two or more different HLA alleles.

The present invention defines positions within a motif enabling the selection of peptides that will bind efficiently to more than one HLA-A, HLA-B or HLA-C alleles. Epitopes possessing the motif of the immunogenic peptides have been identified on potential target antigens including hepatitis B core and surface antigens (HBVc, HBVs), hepatitis C antigens, Epstein-Barr virus antigens, and human immunodeficiency type-1 virus (HIV1). Thus, the invention further provides immunogenic peptides comprising sequences of target antigens.

The peptides of the invention are useful in pharmaceutical compositions for both in vivo and ex vivo therapeutic and diagnostic applications.

### Definitions

The term “peptide” is used interchangeably with “oligopeptide” in the present specification to designate a series of residues, typically L-amino acids, connected one to the other typically by peptide bonds between the alpha-amino and carbonyl groups of adjacent amino acids. The oligopeptides of the invention are less than about 15 residues in length and usually consist of between about 8 and about 11 residues, preferably 9 or 10 residues.

An “immunogenic peptide” is a peptide which comprises an allele-specific motif such that the peptide will bind an MHC molecule and induce a CTL response. Immunogenic peptides of the invention are capable of binding to an appropriate HLA molecule and inducing a cytotoxic T cell response against the antigen from which the immunogenic peptide is derived.

A “conserved residue” is a conserved amino acid occupying a particular position in a peptide motif typically one where the MHC structure may provide a contact point with the immunogenic peptide. One to three, typically two, conserved residues within a peptide of defined length defines a motif for an immunogenic peptide. These residues are typically in close contact with the peptide binding groove, with their side chains buried in specific pockets of the groove itself.

The term “motif” refers to the pattern of residues in a peptide of defined length, usually about 8 to about 11 amino acids, which is recognized by a particular MHC allele. The peptide motifs are typically different for each human MHC allele.

The term “supermotif” refers to motifs that, when present in an immunogenic peptide, allow the peptide to bind more than one HLA antigen. The supermotif preferably is recognized by at least one HLA allele having a wide distribution in the human population, preferably recognized by at least two alleles, more preferably recognized by at least three alleles, and most preferably recognized by more than three alleles.

The phrases “isolated” or “biologically pure” refer to material which is substantially or essentially free from components which normally accompany it as found in its native state. Thus, the peptides of this invention do not contain materials normally associated with their in situ environment, e.g., MHC I molecules on antigen presenting cells. Even where a protein has been isolated to a homogenous or dominant band, there are trace contaminants in the range of 5-10% of native protein which co-purify with the desired protein. Isolated peptides of this invention do not contain such endogenous co-purified protein.

The term “residue” refers to an amino acid or amino acid mimetic incorporated in an oligopeptide by an amide bond or amide bond mimetic.

### BRIEF DESCRIPTION OF THE DRAWINGS

FIG. 1 shows binding motifs for peptides capable of binding HLA alleles sharing the B7-like specificity.

FIG. 2 shows the B7-like cross-reactive motif.

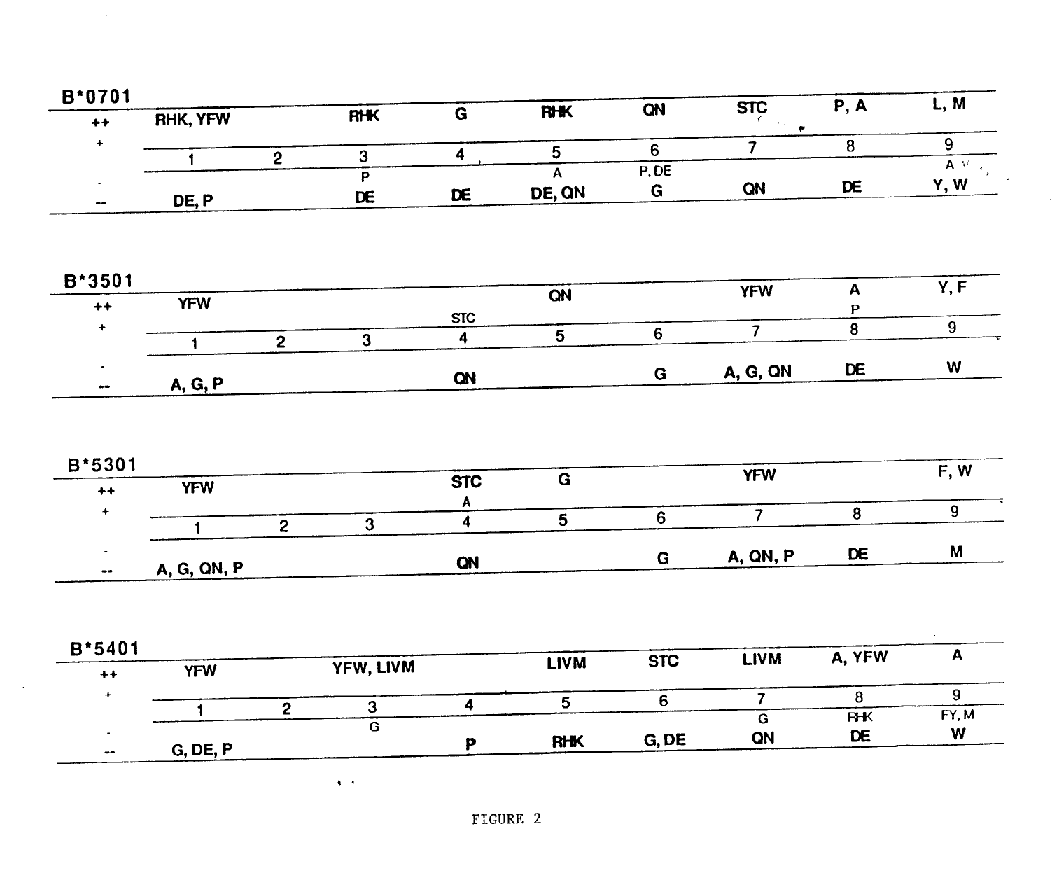

### DESCRIPTION OF THE PREFERRED EMBODIMENTS

The present invention relates to the determination of allele-specific peptide motifs for human Class I MHC (sometimes referred to as HLA) allele subtypes. In particular, the invention provides motifs that are common to peptides bound by more than one HLA allele. By a combination of motif identification and MHC-peptide interaction studies, peptides useful for peptide vaccines have been identified.

Following the methods described in the copending applications noted above, certain peptides capable of binding at multiple HLA alleles which possess a common motif have been identified. The motifs of those peptides can be characterized as follows: N-XPXXXXXX(AVILM)-C; N-XPXXXXXXX(AVILM)-C; N-XPXXXXXX(FWY)-C; and N-XPXXXXXXX(FWY)-C. Motifs that are capable of binding at multiple alleles are referred to here as “supermotifs.” The particular supermotifs above are specifically called “B7-like-supermotifs.”

Immunogenic peptides of the invention are typically identified using a computer to scan the amino acid sequence of a desired antigen for the presence of the supermotifs. Examples of antigens include viral antigens and antigens associated with cancer. An antigen associated with cancer is an antigen, such as a melanoma antigen, that is characteristic of (i.e., expressed by) cells in a malignant tumor but not normally expressed by healthy cells. Examples of suitable antigens particularly include hepatitis B core and surface antigens (HBVc, HBVs) hepatitis C antigens, Epstein-Barr virus antigens, and human immunodeficiency virus (HIV) antigens, and also include prostate specific antigen (PSA), melanoma antigens (e.g., MAGE-1), and human papilloma virus (HPV) antigens; this list is not intended to exclude other sources of antigens.

Peptides comprising the supermotif sequences, including those found in proteins from potential antigenic sources are synthesized and then tested for their ability to bind to the appropriate MHC molecules in a variety of assays. The assays may use, for example, purified class I molecules and radioiodonated peptides. Alternatively, binding to cells expressing empty class I molecules can be detected by, for instance, immunofluorescent staining and flow microfluorimetry. Those peptides that bind to the class I molecule may be further evaluated for their ability to serve as targets for CTLs derived from infected or immunized individuals, as well as for their capacity to induce primary in vitro or in vivo CTL responses that can give rise to CTL populations capable of reacting with virally infected target cells or tumor cells as therapeutic agents.

Recent evidence suggests however, that high affinity MHC binders might be, in most instances, immunogenic, suggesting that peptide epitopes might be selected on the basis of MHC binding alone.

Peptides comprising the supermotif sequences can be identified, as noted above, by screening potential antigenic sources. Useful peptides can also be identified by synthesizing peptides with systematic or random substitution of the variable residues in the supermotif, and testing them according to the assays provided. As demonstrated below, it is useful to refer to the sequences of the target HLA molecule, as well.

The nomenclature used to describe peptide compounds follows the conventional practice wherein the amino group is presented to the left (the N-terminus) and the carboxyl group to the right (the C-terminus) of each amino acid residue. In the formulae representing selected specific embodiments of the present invention, the amino- and carboxyl-terminal groups, although not specifically shown, are in the form they would assume at physiologic Ph values, unless otherwise specified. In the amino acid structure formulae, each residue is generally represented by standard three letter or single letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G. The letter X in a motif represents any of the 20 amino acids found in Table 1, as well non-naturally occurring amino acids or amino acid mimetics. Brackets surrounding more than one amino acid indicates that the motif includes any one of the amino acids. For example, the supermotif “N-XPXXXXXX(AVILM)-C” includes each of the following peptides: N-XPXXXXXXA-C, N-XPXXXXXXV-C, N-XPXXXXXXI-C, N-XPXXXXXXL-C, and N-XPXXXXXXM-C.

For peptide-based vaccines, the peptides of the present invention preferably comprise a motif (Table 2) shows the distribution of certain HLA alleles in human populations.

TABLE 1

| Original Residue | Exemplary Substitution |
|---|---|
| Ala | ser |
| Arg | lys |
| Asn | gln |
| Asp | glu |
| Cys | ser |
| Gln | asn |
| Glu | asp |
| Gly | pro |
| His | arg; lys |
| Ile | leu; val; met |
| Leu | ile; val; met |
| Lys | arg |
| Met | leu; ile; val |
| Phe | tyr; trp |
| Ser | thr |
| Thr | ser |
| Trp | tyr; phe |
| Tyr | trp; phe |
| Val | ile; leu; met |

TABLE 2
Summary of Population Coverage by Currently Available Assays

| | | | Phenotypic (Allelic) Frequency | | | | |
|---|---|---|---|---|---|---|---|
| Antigen | HLA Allele | Cell Line(s) | Caucasian | Negro | Japanese | Chinese | Hispanic |
| A1 | A*0101 | Steinlin | 28.6 | 10.1 | 1.4 | 9.2 | 10.1 |
| A2.1 | A*0201 | JY | 45.8 | 30.3 | 42.4 | 54.0 | 43.0 |
| A3.2 | A*0301 | GM3107 | 20.6 | 16.3 | 1.2 | 7.1 | 14.8 |
| A11 | A*1101 | BVR | 9.9 | 3.8 | 19.7 | 33.1 | 7.3 |
| A24 | A*2401 | KT3 | 16.8 | 8.8 | 58.1 | 32.9 | 26.7 |
| All A | | | 88.9 | 59.8 | 91.6 | 94.6 | 80.2 |
| B7 | B*0701 | GM3107 | 17.7 | 15.5 | 9.6 | 6.9 | 11.8 |
| B8 | B*0801 | Steinlin | 18.1 | 6.3 | 0.0 | 3.6 | 9.0 |
| B27 | B*2705 | LG2 | 7.5 | 2.6 | 0.8 | 3.4 | 4.9 |
| B35 | B*3503 | BHM | 15.4 | 14.8 | 15.4 | 9.8 | 28.1 |
| B54 | B*5401 | KT3 | 0.0 | 0.0 | 12.4 | 8.6 | 0.0 |
| All B | | | 51.9 | 36.5 | 35.6 | 30.2 | 48.7 |
| Cw6 | Cw0601 | C1R | 17.6 | 13.7 | 2.2 | 19.0 | 12.2 |
| TOTAL | | | 95.7 | 76.5 | 94.7 | 96.6 | 91.0 |

For assays of peptide-HLA interactions (e.g., quantitative binding assays) cells with defined MHC molecules are useful. A large number of cells with defined MHC molecules, particularly MHC Class I molecules, are known and readily available. For example, human EBV-transformed B cell lines have been shown to be excellent sources for the preparative isolation of class I and class II MHC molecules. Well-characterized cell lines are available from private and commercial sources, such as American Type Culture Collection (“Catalogue of Cell Lines and Hybridomas,” 6th edition (1988) Rockville, Md., U.S.A.); National Institute of General Medical Sciences 1990/1991 Catalog of Cell Lines (NIGMS) Human Genetic Mutant Cell Repository, Camden, N.J.; and ASHI Repository, Brigham and Women's Hospital, 75 Francis Street, Boston, Mass. 02115. Cell lines suitable as sources for various HLA-A alleles are described in the copending applications. Table 3 lists some B cell lines suitable for use as sources for HLA-B and HLA-C alleles, which are particularly useful in the present invention. All of these cell lines can be grown in large batches and are therefore useful for large scale production of MHC molecules. One of skill will recognize that these are merely exemplary cell lines and that many other cell sources can be employed.

TABLE 3
HUMAN CELL LINES (HLA-B and HLA-C SOURCES)

| HLA-B allele | B cell line |
|---|---|
| B1801 | DVCAF |
| B3503 | EHM |
| B0701 | GM3107 |
| B1401 | LWAGS |
| B5101 | KAS116 |
| B5301 | AMAI |
| B0801 | MAT |
| B2705 | LG2 |
| B5401 | KT3 |
| B1302 | CBUF |
| B4403 | PITOUT |
| B3502 | TISI |
| B3501 | BUR |
| B4001 | LB |
| HLA-C allele | B cell line |
| Cw0601 | C1R |

In the typical case, immunoprecipitation is used to isolate the desired allele. A number of protocols can be used, depending upon the specificity of the antibodies used. For example, allele-specific mAb reagents can be used for the affinity purification of the HLA-A, HLA-B, and HLA-C molecules. Monoclonal antibodies available for isolating various HLA molecules include those listed in Table 4. Affinity columns prepared with these mAbs using standard techniques are used to purify the respective HLA allele products.

TABLE 4
ANTIBODY REAGENTS

| anti-HLA | Name |
|---|---|
| HLA-A2 | BB7.2 |
| HLA-A1 | 12/18 |
| HLA-A3 | GAPA3 (ATCC, HB122) |
| HLA-11, 24.1 | A11.1M (ATCC, HB164) |
| HLA-A, B, C | W6/32 (ATCC, HB95) |
| monomorphic | B9.12.1 |
| HLA-B, C | B.1.23.2 |
| monomorphic | |

The capacity to bind MHC Class I molecules is measured in a variety of different ways. One means is a Class I molecular binding assay as described in Example 2, below. Other alternatives described in the literature include inhibition of antigen presentation (Sette, et al., J. Immunol. 141:3893 (1991)), in vitro assembly assays (Townsend, et al., Cell 62:285 (1990)), and FACS based assays using mutated cells, such as RMA.S (Melief, et al., Eur. J. Immunol. 21:2963 (1991)).

Next, peptides that test positive in the MHC class I binding assay are assayed for the ability of the peptides to induce specific CTL responses in vitro. For instance, antigen-presenting cells that have been incubated with a peptide can be assayed for the ability to induce CTL responses in responder cell populations. Antigen-presenting cells can be normal cells such as peripheral blood mononuclear cells or dendritic cells (Inaba, et al., J. Exp. Med. 166:182 (1987); Boog, Eur. J. Immunol. 18:219 (1988)). Alternatively, transgenic mice comprising an appropriate HLA transgene can be used to assay the ability of a peptide to induce a response in cytotoxic T lymphocytes essentially as described in copending U.S. patent application Ser. No. 08/205,713.

Alternatively, mutant mammalian cell lines that are deficient in their ability to load class I molecules with internally processed peptides, such as the mouse cell lines RMA-S (Kärre, et al. Nature, 319:675 (1986); Ljunggren, et al., Eur. J. Immunol. 21:2963-2970 (1991)), and the human T cell hybridoma, T-2 (Cerundolo, et al., Nature 345:449-452 (1990)) and which have been transfected with the appropriate human class I genes are conveniently used, when peptide is added to them, to test for the capacity of the peptide to induce in vitro primary CTL responses. Other eukaryotic cell lines which could be used include various insect cell lines such as mosquito larvae (ATCC cell lines CCL 125, 126, 1660, 1591, 6585, 6586), silkworm (ATTC CRL 8851), armyworm (ATCC CRL 1711), moth (ATCC CCL 80) and Drosophila cell lines such as a Schneider cell line (see Schneider J. Embryol. Exp. Morphol. 27:353-365 [1927]).

Peripheral blood lymphocytes are conveniently isolated following simple venipuncture or leukapheresis of normal donors or patients and used as the responder cell sources of CTL precursors. In one embodiment, the appropriate antigen-presenting cells are incubated with 10-100 μM of peptide in serum-free media for 4 hours under appropriate culture conditions. The peptide-loaded antigen-presenting cells are then incubated with the responder cell populations in vitro for 7 to 10 days under optimized culture conditions. Positive CTL activation can be determined by assaying the cultures for the presence of CTLs that kill radiolabeled target cells, both specific peptide-pulsed targets as well as target cells expressing endogenously processed form of the relevant virus or tumor antigen from which the peptide sequence was derived.

Specificity and MHC restriction of the CTL is determined by testing against different peptide target cells expressing appropriate or inappropriate human MHC class I. The peptides that test positive in the MHC binding assays and give rise to specific CTL responses are referred to herein as immunogenic peptides.

The immunogenic peptides can be prepared synthetically, or by recombinant DNA technology. Although the peptide will preferably be substantially free of other naturally occurring host cell proteins and fragments thereof, in some embodiments the peptides can be synthetically conjugated to native fragments or particles.

The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

Desirably, the peptide will be as small as possible while still maintaining substantially all of the biological activity of the large peptide. When possible, it may be desirable to optimize peptides of the invention to a length of 9 or 10 amino acid residues, commensurate in size with endogenously processed viral peptides or tumor cell peptides that are bound to MHC class I molecules on the cell surface.

Peptides having the desired activity may be modified as necessary to provide certain desired attributes, e.g., improved pharmacological characteristics, while increasing or at least retaining substantially all of the biological activity of the unmodified peptide to bind the desired MHC molecule and activate the appropriate T cell. For instance, the peptides may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany and Merrifield, The Peptides, Gross and Meienhofer, eds. (N.Y., Academic Press), pp. 1-284 (1979); and Stewart and Young, Solid Phase Peptide Synthesis, (Rockford, Ill., Pierce), 2d Ed. (1984), incorporated by reference herein.

The peptides can also be modified by extending or decreasing the compound's amino acid sequence, e.g., by the addition or deletion of amino acids. The peptides or analogs of the invention can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-protein amino acids as well, such as β-±-δ-amino acids, as well as many derivatives of L-α-amino acids.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place. Such substitutions generally are made in accordance with Table 1 when it is desired to finely modulate the characteristics of the peptide.

Substantial changes in function (e.g., affinity for MHC molecules or T cell receptors) are made by selecting substitutions that are less conservative than those in Table 1, i.e., selecting residues that differ more significantly in their effect on maintaining (a) the structure of the peptide backbone in the area of the substitution, for example as a sheet or helical conformation, (b) the charge or hydrophobicity of the molecule at the target site or (c) the bulk of the side chain. The substitutions which in general are expected to produce the greatest changes in peptide properties will be those in which (a) hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g., glycine.

The peptides may also comprise isosteres of two or more residues in the immunogenic peptide. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the α-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

Modifications of peptides with various amino acid mimetics or D-amino acids, for instance at the N- or C-termini, are particularly useful in increasing the stability of the peptide in vivo. Stability can be assayed in a number of ways. For instance, peptidases and various biological media, such as human plasma and serum, have been used to test stability. See, e.g., Verhoef et al., Eur. J. Drug Metab. Pharmacokin. 11:291-302 (1986). Half life of the peptides of the present invention is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

The peptides of the present invention or analogs thereof which have CTL stimulating activity may be modified to provide desired attributes other than improved serum half life. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions and may have linear or branched side chains. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the CTL peptide may be linked to the T helper peptide without a spacer.

The immunogenic peptide may be linked to the T helper peptide either directly or via a spacer either at the amino or carboxy terminus of the CTL peptide. The amino terminus of either the immunogenic peptide or the T helper peptide may acylated. Exemplary T helper peptides include tetanus toxoid 830-843, influenza 307-319, malaria circumsporozoite 382-398 and 378-389.

In some embodiments it may be desirable to include in the pharmaceutical compositions of the invention at least one component which primes CTL. Lipids have been identified as agents capable of priming CTL in vivo against viral antigens. For example, palmitic acid residues can be attached to the alpha and epsilon amino groups of a Lys residue and then linked, e.g., via one or more linking residues such as Gly, Gly-Gly-, Ser, Ser-Ser, or the like, to an immunogenic peptide. The lipidated peptide can then be injected directly in a micellar form, incorporated into a liposome or emulsified in an adjuvant, e.g., incomplete Freund's adjuvant. In a preferred embodiment a particularly effective immunogen comprises palmitic acid attached to alpha and epsilon amino groups of Lys, which is attached via linkage, e.g., Ser-Ser, to the amino terminus of the immunogenic peptide.

As another example of lipid priming of CTL responses, E. coli lipoproteins, such as tripalmitoyl-S-glycerylcysteinlyseryl-serine (P₃CSS) I can be used to prime virus specific CTL when covalently attached to an appropriate peptide. See, Deres et al., Nature 342:561-564 (1989), incorporated herein by reference. Peptides of the invention can be coupled to P₃CSS, for example, and the lipopeptide administered to an individual to specifically prime a CTL response to the target antigen. Further, as the induction of neutralizing antibodies can also be primed with P₃CSS conjugated to a peptide which displays an appropriate epitope, the two compositions can be combined to more effectively elicit both humoral and cell-mediated responses to infection.

In addition, additional amino acids can be added to the termini of a peptide to provide for ease of linking peptides one to another, for coupling to a carrier support, or larger peptide, for modifying the physical or chemical properties of the peptide or oligopeptide, or the like. Amino acids such as tyrosine, cysteine, lysine, glutamic or aspartic acid, or the like, can be introduced at the C- or N-terminus of the peptide or oligopeptide. Modification at the C terminus in some cases may alter binding characteristics of the peptide. In addition, the peptide or oligopeptide sequences can differ from the natural sequence by being modified by terminal-NH₂ acylation, e.g., by alkanoyl (C₁-C₂₀) or thioglycolyl acetylation, terminal-carboxyl amidation, e.g., ammonia, methylamine, etc. In some instances these modifications may provide sites for linking to a support or other molecule.

The peptides of the invention can be prepared in a wide variety of ways. Because of their relatively short size, the peptides can be synthesized in solution or on a solid support in accordance with conventional techniques. Various automatic synthesizers are commercially available and can be used in accordance with known protocols. See, for example, Stewart and Young, Solid Phase Peptide Synthesis, 2d. ed., Pierce Chemical Co. (1984), supra.

Alternatively, recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1982), which is incorporated herein by reference. Thus, fusion proteins which comprise one or more peptide sequences of the invention can be used to present the appropriate T cell epitope.

As the coding sequence for peptides of the length contemplated herein can be synthesized by chemical techniques, for example, the phosphotriester method of Matteucci et al., J. Am. Chem. Soc. 103:3185 (1981), modification can be made simply by substituting the appropriate base(s) for those encoding the native peptide sequence. The coding sequence can then be provided with appropriate linkers and ligated into expression vectors commonly available in the art, and the vectors used to transform suitable hosts to produce the desired fusion protein. A number of such vectors and suitable host systems are now available. For expression of the fusion proteins, the coding sequence will be provided with operably linked start and stop codons, promoter and terminator regions and usually a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts. Of course, yeast or mammalian cell hosts may also be used, employing suitable vectors and control sequences.

The peptides of the present invention and pharmaceutical and vaccine compositions thereof are useful for administration to mammals, particularly humans, to treat and/or prevent viral infection and cancer. Examples of diseases which can be treated using the immunogenic peptides of the invention include prostate cancer, hepatitis B, hepatitis C, AIDS, renal carcinoma, cervical carcinoma, lymphoma, CMV and condlyloma acuminatum.

For pharmaceutical compositions, the immunogenic peptides of the invention are administered to an individual already suffering from cancer or infected with the virus of interest. Those in the incubation phase or the acute phase of infection can be treated with the immunogenic peptides separately or in conjunction with other treatments, as appropriate. In therapeutic applications, compositions are administered to a patient in an amount sufficient to elicit an effective CTL response to the virus or tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 5000 μg of peptide for a 70 kg patient, followed by boosting dosages of from about 1.0 μg to about 1000 μg of peptide pursuant to a boosting regimen over weeks to months depending upon the patient's response and condition by measuring specific CTL activity in the patient's blood. It must be kept in mind that the peptides and compositions of the present invention may generally be employed in serious disease states, that is, life-threatening or potentially life threatening situations. In such cases, in view of the minimization of extraneous substances and the relative nontoxic nature of the peptides, it is possible and may be felt desirable by the treating physician to administer substantial excesses of these peptide compositions.

For therapeutic use, administration should begin at the first sign of viral infection or the detection or surgical removal of tumors or shortly after diagnosis in the case of acute infection. This is followed by boosting doses until at least symptoms are substantially abated and for a period thereafter. In chronic infection, loading doses followed by boosting doses may be required.

Treatment of an infected individual with the compositions of the invention may hasten resolution of the infection in acutely infected individuals. For those individuals susceptible (or predisposed) to developing chronic infection the compositions are particularly useful in methods for preventing the evolution from acute to chronic infection. Where the susceptible individuals are identified prior to or during infection, for instance, as described herein, the composition can be targeted to them, minimizing need for administration to a larger population.

The peptide compositions can also be used for the treatment of chronic infection and to stimulate the immune system to eliminate virus-infected cells in carriers. It is important to provide an amount of immuno-potentiating peptide in a formulation and mode of administration sufficient to effectively stimulate a cytotoxic T cell response. Thus, for treatment of chronic infection, a representative dose is in the range of about 1.0 μg to about 5000 μg, preferably about 5 μg to 1000 μg for a 70 kg patient per dose. Immunizing doses followed by boosting doses at established intervals, e.g., from one to four weeks, may be required, possibly for a prolonged period of time to effectively immunize an individual. In the case of chronic infection, administration should continue until at least clinical symptoms or laboratory tests indicate that the viral infection has been eliminated or substantially abated and for a period thereafter.

The pharmaceutical compositions for therapeutic treatment are intended for parenteral, topical, oral or local administration. Preferably, the pharmaceutical compositions are administered parenterally, e.g., intravenously, subcutaneously, intradermally, or intramuscularly. Thus, the invention provides compositions for parenteral administration which comprise a solution of the immunogenic peptides dissolved or suspended in an acceptable carrier, preferably an aqueous carrier. A variety of aqueous carriers may be used, e.g., water, buffered water, 0.4% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

In some embodiments it may be desirable to include in the pharmaceutical composition at least one component which enhances priming of CTL. Lipids have been identified as agents capable of enhancing priming of CTL in vivo against viral antigens. For example, palmitic acid residues can be attached to the alpha and epsilon amino groups of a Lys residue and then linked, e.g., typically via one or more linking residues such as Gly, Gly-Gly-, Ser, Ser-Ser, or the like, to a synthetic peptide which comprises a class I-restricted CTL epitope. The lipidated peptide can be administered in saline or incorporated into a liposome emulsified in an adjuvant, e.g., incomplete Freund's adjuvant. In a preferred embodiment a particularly effective immunogen comprises palmitic acid attached to alpha and epsilon amino groups of Lys, which is attached via linkage, e.g., Ser-Ser, to the amino terminus of a class I restricted peptide having T cell determinants, such as those peptides described herein as well as other peptides which have been identified as having such determinants.

As another example of lipid priming of CTL responses, E. coli lipoprotein, such as tripalmitoyl-S-glycerylcysteinly-seryl-serine (p₃CSS), can be used to prime virus specific CTL when covalently attached to an appropriate peptide. See, Deres et al., Nature 342:561-564 (1989), incorporated herein by reference. Peptides of the invention can be coupled to P₃CSS, for example, and the lipopeptide administered to an individual to specifically prime a CTL. Further, as the induction of neutralizing antibodies can also be primed with P₃CSS conjugated to a peptide which displays an appropriate epitope, the two compositions can be combined to more effectively elicit both humoral and cell-mediated responses to viral infection.

The concentration of CTL stimulatory peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The peptides of the invention may also be administered via liposomes, which serve to target the peptides to a particular tissue, such as lymphoid tissue, or targeted selectively to infected cells, as well as increase the half-life of the peptide composition. Liposomes include emulsions, foams, micelles, insoluble monolayers, liquid crystals, phospholipid dispersions, lamellar layers and the like. In these preparations the peptide to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired peptide of the invention can be directed to the site of lymphoid cells, where the liposomes then deliver the selected therapeutic/immunogenic peptide compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9:467 (1980), U.S. Pat. Nos. 4,235,871, 4,501,728, 4,837,028, and 5,019,369, incorporated herein by reference.

For targeting to the immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included, as desired, as with, e.g., lecithin for intranasal delivery.

In another aspect the present invention is directed to vaccines which contain as an active ingredient an immunogenically effective amount of an immunogenic peptide as described herein. The peptide(s) may be introduced into a host, including humans, linked to its own carrier or as a homopolymer or heteropolymer of active peptide units. Such a polymer has the advantage of increased immunological reaction and, where different peptides are used to make up the polymer, the additional ability to induce antibodies and/or CTLs that react with different antigenic determinants of the virus or tumor cells. Useful carriers are well known in the art, and include, e.g., thyroglobulin, albumins such as human serum albumin, tetanus toxoid, polyamino acids such as poly(oysine:glutamic acid), influenza, hepatitis B virus core protein, hepatitis B virus recombinant vaccine and the like. The vaccines can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, or alum are materials well known in the art. And, as mentioned above, CTL responses can be primed by conjugating peptides of the invention to lipids, such as P₃CSS. Upon immunization with a peptide composition as described herein, via injection, aerosol, oral, transdermal or other route, the immune system of the host responds to the vaccine by producing large amounts of CThs specific for the desired antigen, and the host becomes at least partially immune to later infection, or resistant to developing chronic infection.

Vaccine compositions containing the peptides of the invention are administered to a patient susceptible to or otherwise at risk of viral infection or cancer to elicit an immune response against the antigen and thus enhance the patient's own immune response capabilities. Such an amount is defined to be an “immunogenically effective dose.” In this use, the precise amounts again depend on the patient's state of health and weight, the mode of administration, the nature of the formulation, etc., but generally range from about 1.0 μg to about 5000 μg per 70 kilogram patient, more commonly from about 10 μg to about 500 μg mg per 70 kg of body weight.

In some instances it may be desirable to combine the peptide vaccines of the invention with vaccines which induce neutralizing antibody responses to the virus of interest, particularly to viral envelope antigens.

For therapeutic or immunization purposes, the peptides of the invention can also be expressed by attenuated viral hosts, such as vaccinia or fowlpox. This approach involves the use of vaccinia virus as a vector to express nucleotide sequences that encode the peptides of the invention. Upon introduction into an acutely or chronically infected host or into a non-infected host, the recombinant vaccinia virus expresses the immunogenic peptide, and thereby elicits a host CTL response. Vaccinia vectors and methods useful in immunization protocols are described in, e.g., U.S. Pat. No. 4,722,848, incorporated herein by reference. Another vector is BCG (Bacille Calmette Guerin). BCG vectors are described in Stover et al. (Nature 351:456-460 (1991)) which is incorporated herein by reference. A wide variety of other vectors useful for therapeutic administration or immunization of the peptides of the invention, e.g., Salmonella typhi vectors and the like, will be apparent to those skilled in the art from the description herein.

Antigenic peptides may be used to elicit CTL ex vivo, as well. The resulting CTL, can be used to treat chronic infections (viral or bacterial) or tumors in patients that do not respond to other conventional forms of therapy, or will not respond to a peptide vaccine approach of therapy. Ex vivo CTL responses to a particular pathogen (infectious agent or tumor antigen) are induced by incubating in tissue culture the patient's CTL precursor cells (CTLp) together with a source of antigen-presenting cells (APC) and the appropriate immunogenic peptide. After an appropriate incubation time (typically 1-4 weeks), in which the CTLp are activated and mature and expand into effector CTL, the cells are infused back into the patient, where they will destroy their specific target cell (an infected cell or a tumor cell).

The peptides may also find use as diagnostic reagents. For example, a peptide of the invention may be used to determine the susceptibility of a particular individual to a treatment regimen which employs the peptide or related peptides, and thus may be helpful in modifying an existing treatment protocol or in determining a prognosis for an affected individual. In addition, the peptides may also be used to predict which individuals will be at substantial risk for developing chronic infection.

The following examples are offered by way of illustration, not by way of limitation.

### EXAMPLE 1

### Class I Antigen Isolation

Isolated MHC molecules were used in a quantitative binding assay to identify the specificity and avidity of peptide-HLA interactions. Purification of HLA-A, HLA-B and HLA-C antigens were carried out by essentially similar methods, using cells and antibodies chosen as appropriate for the desired HLA molecule. Briefly, the cells bearing the appropriate allele were grown in large batches (6-8 liters yielding ˜5×10⁹ cells), harvested by centrifugation and washed. All cell lines were maintained in RPMI 1640 media (Sigma) supplemented with 10% fetal bovine serum (FBS) and antibiotics.

For large-scale cultures, cells were grown in roller bottle culture in RPMI 1640 with 10% FBS or with 10% horse serum and antibiotics. Cells were harvested by centrifugation at 1500 RPM IEC-CRU5000 centrifuge with 259 rotor and washed three times with phosphate-buffered saline (PBS)(0.01 M PO₄, 0.154 M NaCl, pH 7.2). Cells were pelleted and stored at −70° C. or treated with detergent lysing solution to prepare detergent lysates. Cell lysates were prepared by the addition of stock detergent solution [1% NP-40 (Sigma) or Renex 30 (Accurate Chem. Sci. Corp., Westbury, N.Y. 11590), 150 mM NaCl, 50 mM Tris, pH 8.0] to the cell pellets (previously counted) at a ratio of 50-100×10⁶ cells per ml detergent solution. A cocktail of protease inhibitors was added to the premeasured volume of stock detergent solution immediately prior to the addition to the cell pellet. Addition of the protease inhibitor cocktail produced final concentrations of the following: phenylmethylsulfonyl fluoride (PMSF), 2 mM; aprotinin, 5 μg/ml; leupeptin, 10 μg/ml; pepstatin, 10μg/ml; iodoacetamide, 100 μM; and EDTA, 3 ng/ml. Cell lysis was allowed to proceed at 4° C. for 1 hour with periodic mixing. Routinely 5-10×10⁹ cells were lysed in 50-100 ml of detergent solution. The lysate was clarified by centrifugation at 15,000× g for 30 minutes at 4° C. and subsequent passage of the supernatant fraction through a 0.2 μ filter unit (Nalgene). Cell lines used for HLA-B and -C isolations are provided in Table 3.

The HLA antigen purification was achieved using affinity columns prepared with mAb-conjugated Sepharose beads. For antibody production, cells were grown in RPMI with 10% FBS in large tissue culture flasks (Corning 25160-225). Antibodies were purified from clarified tissue culture medium by ammonium sulfate fractionation followed by affinity chromatography on protein-A-Sepharose (Sigma). Briefly, saturated ammonium sulfate was added slowly with stirring to the tissue culture supernatant to 45% (volume to volume) overnight at 4° C. to precipitate the immunoglobulins. The precipitated proteins were harvested by centrifugation at 10,000× g for 30 minutes. The precipitate was then dissolved in a minimum volume of PBS and transferred to dialysis tubing (Spectro/Por 2, Mol. wt. cutoff 12,000-14,000, Spectum Medical Ind.). Dialysis was against PBS (≧20 times the protein solution volume) with 4-6 changes of dialysis buffer over a 24-48 hour period at 4° C. The dialyzed protein solution was clarified by centrifugation (10,000× g for 30 minutes) and the pH of the solution adjusted to pH 8.0 with 1N NaOH. Protein-A-Sepharose (Sigma) was hydrated according to the manufacturer's instructions, and a protein-A-Sepharose column was prepared. A column of 10 ml bed volume typically binds 50-100 mg of mouse IgG.

The protein sample was loaded onto the protein-A-Sepharose column using a peristaltic pump for large loading volumes or by gravity for smaller volumes (<100 ml). The column was washed with several volumes of PBS, and the eluate was monitored at A280 in a spectrophotometer until base line was reached. The bound antibody was eluted using 0.1 M citric acid at suitable pH (adjusted to the appropriate pH 2 with 1N NaOH). For mouse IgG-1 pH 6.5 was used for IgG2a pH 4.5 was used and for IgG2b and IgG3 pH 3.0 was used. 2 M Tris base was used to neutralize the eluate. Fractions containing the antibody (monitored by A280) were pooled, dialyzed against PBS and further concentrated using an Amicon Stirred Cell system (Amicon Model 8050 with YM30 membrane). Antibodies were used for affinity purification of HLA-B and HLA-C molecules are provided in Table 4.

The HLA antigens were purified using affinity columns prepared with mAb-conjugated Sepharose beads. The affinity columns were prepared by incubating protein-A-Sepharose beads (Sigma) with affinity-purified mAb as described above. Five to 10 mg of mAb per ml of bead is the preferred ratio. The mAb bound beads were washed with borate buffer (borate buffer: 100 mM sodium tetraborate, 154 mM NaCl, pH 8.2) until the washes show A280 at based line. Dimethyl pimelimidate (20 mM) in 200 mM triethanolamine was added to covalently crosslink the bound mAb to the protein-A-Sepharose (Schneider et al., J. Biol. Chem. 257:10766 (1982). After incubation for 45 minutes at room temperature on a rotator, the excess crosslinking reagent was removed by washing the beads twice with 10-20 ml of 20 mM ethanolamine, pH 8.2. Between each one the slurry was placed on a rotator for 5 minutes at room temperature. The beads were washed with borate buffer and with PBS plus 0.02% sodium azide.

The cell lysate (5-10×10⁹ cell equivalents) was then slowly passed over a 5-10 ml affinity column (flow rate of 0.1-0.25 ml per minute) to allow the binding of the antigen to the immobilized antibody. After the lysate was allowed to pass through the column, the column was washed sequentially with 20 column volumes of detergent stock solution plus 0.1% sodium dodecyl sulfate, 20 column volumes of 0.5 M NaCl, 20 mM Tris, pH 8.0, and 10 column volumes of 20 mM Tris, pH 8.0. The HLA antigen bound to the mAb was eluted with a basic buffer solution (50 mM diethylamine in water). As an alternative, acid solutions such as 0.15-0.25 M acetic acid were also used to elute the bound antigen. An aliquot of the eluate (1/50) was removed for protein quantification using either a colorimetric assay (BCA assay, Pierce) or by SDS-PAGE, or both. SDS-PAGE analysis was performed as described by Laemmli (Laemmli, U.K., Nature 227:680 (1970)) using known amounts of bovine serum albumin (Sigma) as a protein standard. Allele specific antibodies were used to purify the specific MHC molecule.

### EXAMPLE 2

### Quantitative Binding Assays

Using isolated MHC molecules prepared as described in Example 1, supra, quantitative binding assays were performed. Briefly, indicated amounts of MHC as isolated above were incubated in 0.05% NP40-PBS with ˜5 nM of radiolabeled peptides in the presence of 1-3 λM β₂M and a cocktail of protease inhibitors (final concentrations 1 mM PMSF, 1.3 mM 1.10 Phenanthroline, 73 μM Pepstatin A, 8 mM EDTA, 200 μM N-α-p-tosyl-L-Lysine Chloromethyl ketone). After various times, free and bound peptides were separated by TSK 2000 gel filtration, as described previously in Sette et al., J. Immunol. 148:844 (1992). Peptides were labeled by the use of the Chloramine T method Buus et al., Science 235:1352 (1987).

The various candidate HLA binding peptides were radiolabeled and offered (5-10 nM) to 1 μM purified HLA molecules. After two days at 23° C. in presence of a cocktail of protease inhibitors and 1-3 μM purified human β₂M, the percent of MHC class I bound radioactivity was measured by size exclusion chromatography, as previously described for class II peptide binding assays in Sette et al., in Seminars in Immunology, Vol. 3, Gefter, ed. (W. B. Saunders, Philadelphia, 1991), pp 195-202, which is incorporated herein by reference. Using this protocol, high binding (30-95% of standard peptide binding) was detected in all cases in the presence, but not in the absence, of the relevant HLA allele.

To explore the specificity of binding, we determined whether the binding was inhibitable by excess unlabeled peptide, and if so, what the 50% inhibitory concentration (IC50%) might be. The rationale for this experiment was threefold. First, such an experiment is crucial in order to demonstrate specificity. Second, a sensitive inhibition assay is the most viable alternative for a high throughput quantitative binding assay. Third, inhibition data subjected to Scatchard analysis can give quantitative estimates of the K of interaction and the fraction of receptor molecules capable of binding ligand (% occupancy).

Results of binding assays described here may be expressed in terms of IC50's. Given the conditions in which our assays are run (i.e., limiting MHC and labeled peptide concentrations), these values approximate K_{D} values. It should be noted that IC50 values can change, often dramatically, if the assay conditions are varied, and depending on the particular reagents used (e.g., Class I preparation, etc.). For example, excessive concentrations of MHC will increase the apparent measured IC50 of a given ligand.

An alternative way of expressing the binding data, to avoid these uncertainties, is as a relative value to a reference peptide. The reference peptide is included in every assay. As a particular assay becomes more, or less, sensitive, the IC50's of the peptides tested may change somewhat. However, the binding relative to the reference peptide will not change. For example, in an assay run under conditions such that the IC50 of the reference peptide increases 10-fold, all IC50 values will also shift approximately ten-fold. Therefore, to avoid ambiguities, the assessment of whether a peptide is a good, intermediate, weak, or negative binder should be based on it's IC50, relative to the IC50 of the standard peptide. Reference peptides used in the assays include the following: A1CON1 (YLEPAIAKY), 25 nM for A*0110; HBV core 18-27 F6→Y (FLPSDYFPSV), 4.6 n for A*0201; A3CON1 (KVFPYALINK), 10 nM for A*0301; A3CON1 (KVFPYALINK), 5.9 nM for A*1101; A24CON1 (AYIDNYNKF), 12 nM for A82401; A2.1 signal sequence 5-13 L₇→Y (APRTLVYLL), 4.7 nM for B*0701; HIV gp 586-593 Y₁>F, Q₅>Y (FLKDYQLL), 14 nM for B*0801. Rat 60S (FRYNGLIHR), 6.4 nM for B*2705; B35CON2 (FPFKYAAAF), 4.4 nM B*3503.

If the IC50 of the standard peptide measured in a particular assay is different than that reported in the table then it should be understood that the threshold values used to determine good, intermediate, weak, and negative binders should be modified by a corresponding factor.

### EXAMPLE 3

### Specificity And Cross-Reactivity of HLA Binding

Peptide sequences capable of binding the most common HLA alleles have been identified in previous studies. However, a large number of monospecific epitopes would be required to provide substantial coverage of all ethnic groups. In contrast, the alternative approach of identifying broadly crossreactive motifs (supermotifs) has the potential of covering a similar proportion of the population using just two or three motifs. Table 6 shows a hypothetical population coverage achieved by each of the different motif types or combinations of motif types, using known and predicted motifs.

To explore specificity and cross-reactivity of HLA binding in more detail, a panel of HLA-A and B restricted T cell epitopes was tested for binding in the assays described in Examples 1 & 2, above. It was found (Table SA) that the majority of the peptides were good or intermediate binders to the appropriate restriction element. The binding, in general, was allele-specific. Similar data were obtained with a panel of HLA-B naturally processed peptides (Table 5B), in which it was found that 12 of 12 peptides were good binders to the relevant restriction element. In addition, however, some cross-reactivities were detected, particularly in the case of alleles which had overlapping motifs.

For example, a high degree of cross-reactivity was noted between A3.2 and All (shaded areas, Table SA). The cross-reactivity seen between B7 and B8 with the B8 epitope 1054.05 can be explained by the fact that this peptide has the motif for both B7 and B8. The B7 motif is proline in position 2 and small hydrophobics at the C-terminal. B8 recognized residues with basic charges (RK) in positions 3 and 5, and small hydrophobics at the C-terminal. These data demonstrate that 1) in general, for both the A and B isotypes, the binding is rather specific; and 2) occasional cross-reactivities exist and can usually be explained by either shared motifs or the presence within a single peptide of more than one motif.

TABLE 5A
Relative Binding of HLA-A or B Restricted Peptides

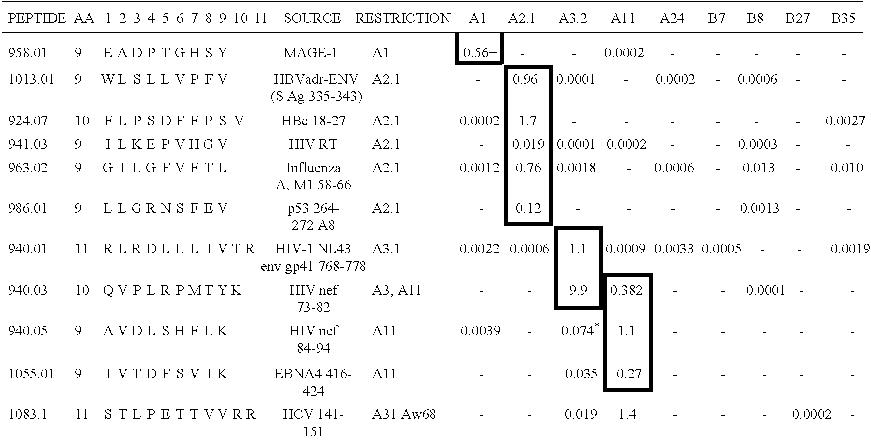

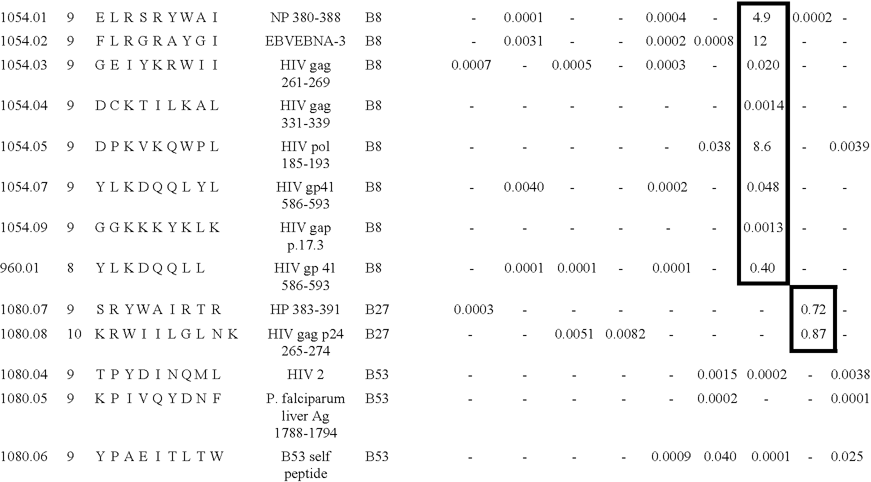

*Shaded areas indicate good or intermediate cross-reactive binding to alleles other than the reported restriction elenent.
+Boxed areas give the binding capacity of epitopes to their reported restriction element.

TABLE 5B
Relative Binding of HLA-B Naturally Processed Peptides

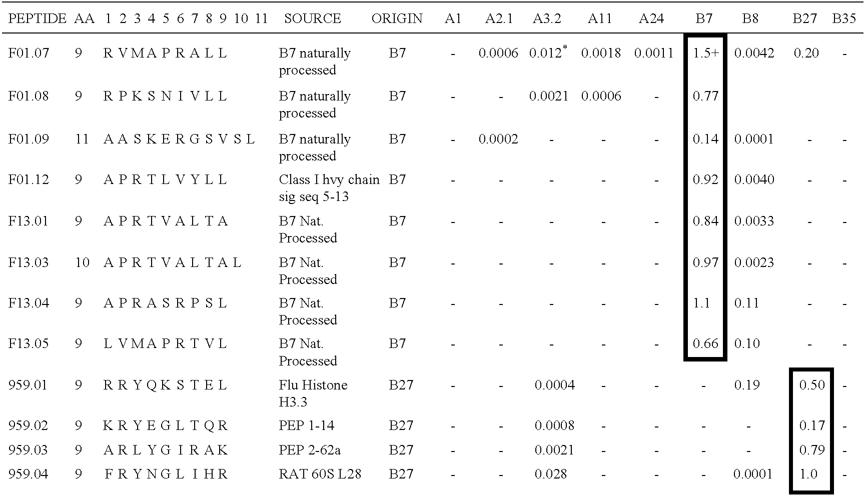

*Shaded areas indicate good or intermediate cross-reactive binding to alleles other than the reported restriction element.
+Boxed areas give the binding capacity of epitopes to their reported restriction element.

The data available thus far have defined a set of motifs which are summarized in Table 6. Three motifs are shared by multiple alleles (identified as types C, D, and F in Table 6). Alleles of type C have hydrophobic residues at position 2 and at the C-terminus; alleles of type D have hydrophobic residues at position 2, with positively charged residues (RK) at the C-terminus; and alleles of type F have proline at position 2, with hydrophobic residues at the C-terminus. Coverage of a significant fraction of the population is achieved by identifying peptides which bind to the alleles listed in Table 6 for the C, D, and F “supermotifs.”

TABLE 6
Compilation of “Known” HLA Motifs

| Motif | B Pocket | F Pocket | | HLA | Cell | Phenotypic (Allelic) Frequency | | | | | Assay |
|---|---|---|---|---|---|---|---|---|---|---|---|
| Type* | Motif | Motif | Antigen | Allele | Line | Caucasian | Negro | Japanese | Chinese | Hispanic | Available |
| A | TS | Y | A1 | A*0101 | Steinlin | 28.6 | 10.1 | 1.4 | 9.2 | 10.1 | yes |
| B | Y | FLI | A24 | A*2401 | KT3 | 16.8 | 8.8 | 58.1 | 32.9 | 26.7 | yes |
| C | VLM | LIV | Aw69.1** | A*6901 | C1R | 0.5 | 0.0 | 0.0 | 0.8 | 0.0 | |
| | LM | LIV | A2.1 | A*0201 | JY | 45.8 | 30.3 | 42.4 | 54.0 | 43.0 | yes |
| All C | | | | | | 46.2 | 30.3 | 42.4 | 54.5 | 43.0 | |
| D | VLM | KR | Aw68.1 | A*6801 | LB | 3.5 | 6.2 | 0.0 | 0.0 | 4.2 | |
| | TV | KR | A11 | A*1101 | BVR | 9.9 | 3.8 | 19.7 | 33.1 | 7.3 | yes |
| | VLH | KR | A3.2 | A*0301 | GM3107 | 20.6 | 16.3 | 1.2 | 7.1 | 14.8 | yes |
| | hydrophobic | KR | Aw31 | A*3101 | | 4.4 | 3.8 | 14.8 | 9.6 | 10.1 | |
| All D | | | | | | 35.9 | 28.6 | 33.9 | 46.3 | 33.9 | |
| E | P | FY | B35 | B*3503 | EHM | 15.4 | 14.8 | 15.4 | 9.8 | 28.1 | yes |
| F | P | LIV(YFW) | B7 | B*0701 | GM3107 | 17.7 | 15.5 | 9.6 | 6.9 | 11.8 | yes |
| | P | LIV | B14 | B*1401 | LWAGS | 7.6 | 6.3 | 0.4 | 0.8 | 12.4 | |
| | P | LIV | B51 | B*5101 | KAS116 | 6.9 | 6.7 | 17.2 | 13.0 | 7.6 | |
| | P | LIVMYFW | B53 | B*5301 | AMAI | 1.6 | 22.6 | 0.2 | 0.0 | 4.2 | |
| | P(R) | LIVMYFW | Cw6 | Cw*0602 | C1R | 17.6 | 13.7 | 2.2 | 19.0 | 12.2 | yes |
| All F | | | | | | 43.9 | 53.6 | 28.0 | 35.3 | 41.7 | |
| G | PK | PK | B27 | B*2705 | LG2 | 7.5 | 2.6 | 0.8 | 3.4 | 4.9 | yes |
| H | RK3, RK5 | LIV | B8 | B*0801 | Steinlin | 18.1 | 6.3 | 0.0 | 3.6 | 9.0 | yes |

*Motifs are grouped as shown below:

| | | |
|---|---|---|
| Motif Type | Position 2 | C-terminus |
| A | sm. polar | tyrosine |
| B | aromatic | hydrophobic |
| C | aliphatic | aliphatic |
| D | aliphatic | basic |
| E | proline | aromatic |
| F | proline | hydrophobic |
| G | basic | basic |
| H | basic/basic | aliphatic |

To date, motifs A-D have been found only in A alleles; F, G, and H are found in B alleles.
**A28 is split into A*6801, A*6802, A*6803, and A*6901. The population distribution of the A28 subtypes was estimated from the overall frequency of the A28 allele and the distribution of the subtypes reported by Fernandez-Vina (Hu. Imm. 33:163)

### EXAMPLE 4

### Prediction of Alleles Binding the Major Motif Supermotifs

Further analysis of the crossreactivity observed between A3, A11, A31, and Aw68 was made by assessing the similarities of these HLA molecules in the residues that make up the B and F binding pockets involved in the interactions with position 2 and the C terminal residue of the peptides which bind these molecules. When this analysis was performed, a high degree of similarity between these alleles becomes evident (see, Matsumura, M. et al. Science 257:927 1992 for a discussion of the structure of the peptide binding pockets in the groove of MHC Class I molecules). Table 7 shows the residues which constitute the F or C-terminal pocket for these alleles. The residues are completely conserved in all four alleles, and experimental data have indicated that each of these alleles recognized basic residues (R,K) at the C-terminus of peptides. B27, an allele which also recognizes basic residues at the C-termini of peptides, differs from A3, A11, A31, and Aw68 by only a single residue, a conservative isoleucine to leucine difference.

TABLE 7
F (C-terminal) Pocket Residues

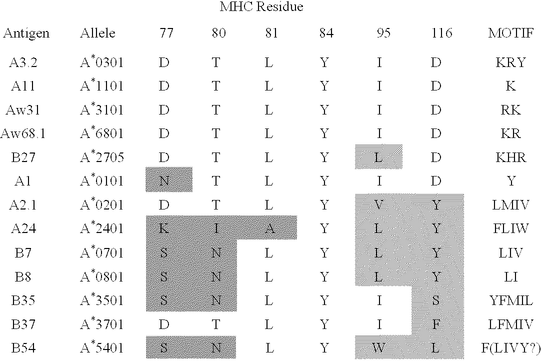

These striking similarities can be contrasted with the sequences of HLA molecules which do not share the basic charge C-terminal motif. Further similarities between A3, A11, A31 and Aw68 are also seen in the B pocket (Table 8), where they also share overlapping motifs (hydrophobics and threonine).

Remarkable motif similarities are demonstrated by the preference of many HLA-B (B7, B14, B35, B51, B53, and B54) and HLA-C (Cw4, Cw6, and Cw7) alleles for proline in position 2. An analysis of the B pocket of the HLA-B alleles is shown in Table 10, and reveals that they all share similar B pockets, having the same or conservatively different (i.e., N/Q) residues in positions 9, 63, 66, and 70. Interestingly, in addition to sharing a motif based on proline in position 2, all of these alleles prefer hydrophobic residues (F of LIV) in position 9.

TABLE 8
B Pocket Comparison of A3-Like Alleles

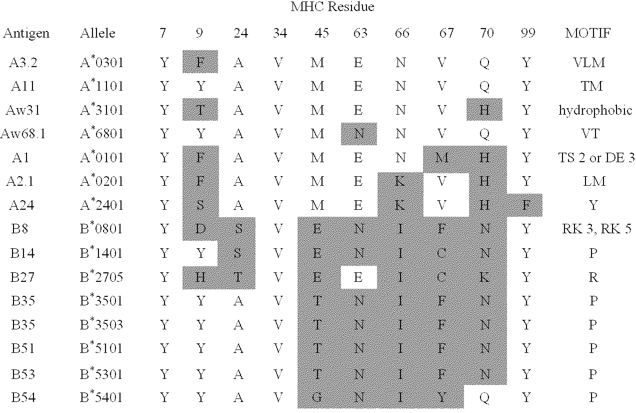

TABLE 9
Predicted Motifs Based on
Structure of B and F Pockets

| | B Pocket | F Pocket | | | |
|---|---|---|---|---|---|
| Motif | Predicted | Predicted | | HLA | Cell |
| Type* | Motif | Motif | Antigen | Allele | Line |
| A | — | Y | B44 | B*4403 | Pitout |
| C | VLM | LIV | Aw68.2 | A*6802 | C1R |
| D | VLM | KR | Aw68.3 | A*6803 | C1R |
| | VLM | KR | A30 | A*3001/3003 | DUCAF, |
| | | | | | LBUF |
| | (LIVMST) | KR | A33 | A*3301 | LWAGS |
| E | P | F | B54 | B*5401 | KT3 |
| F | — | LIVMYFW | Cw3 | Cw*0301 | |
| | PY | LIVMYFW | Cw4 | Cw*0401 | |
| | PY | LIVMYFW | Cw7 | Cw*0701/0702 | C1R, JY |

TABLE 10
B Pocket Comparison of Alleles Preferring Proline in Position 2

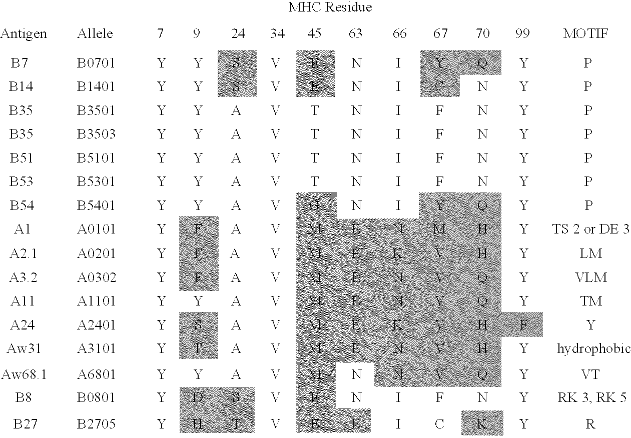

If further alleles could be identified which have motifs fitting the three basic patterns (C, D, and F), it would allow exploitation of crossreactivity using peptides already developed. Crossreactive alleles could be identified by two different approaches. In the first approach, one could establish assays for a large panel of different alleles and empirically determine which motifs fit the various supermotifs. In the second approach, one could attempt to predict a priori crossreactivity based on pocket structure. The analysis discussed above, which compared and contrasted the binding pockets of alleles which share similar B pockets and motifs, or similar F pockets and motifs with alleles which have different motifs, supports the notion that sharing similar pockets will result in the sharing of similar motifs. If this assumption is true, a number of assays for which cell lines are readily available could be explored (Table 9). These alleles all have B and F pockets, which suggests that their motifs might fit into one of the motif types defined in Table 6.

### EXAMPLE 5

### Peptide Binding to B54

To experimentally address the feasibility of increasing allele coverage by a priori selecting alleles which are likely to crossreact, we have examined B54, which is present in about 10% of the Asian population. Sequence analysis of the B pocket of B54 suggested a close similarity to B35, B51, and B53 (Table 10), B54 differing from the other alleles fairly conservatively at three positions. Most interestingly, the polar residues at positions 9, 63, and 70, which are invariable amongst Pro₂ preferring alleles (i.e., alleles to which peptides comprising the B7-like-supermotif bind) and, we speculate, may be crucial for “proline-ness,” were completely invariant. The F pocket of B54 shares the S,N,L triplet at positions 77, 80, and 81 with B7, B8, and B35, and carries a pair of hydrophobic residues at positions 95 and 116, as do these other B alleles. B7, B8, and B35 all prefer peptides with hydrophobic C-terminals.

The analysis discussed above suggested that B54 might recognize peptides carrying a Pro₂-hydrophobic-c-terminal motif (i.e., a B7-like-supermotif). To test this hypothesis, we analyzed whether the B35 binding B35CON2 peptide (Cytel number 1021.05; sequence FPFKYAAAF) could bind to B54. Indeed, excellent binding was detected, with an estimated Kd in the 5 nM range. Thus, a high affinity ligand was selected for B54 based on B and F pocket structural analysis without any previous knowledge of a specific motif. These data illustrate how it may be possible to select, a priori, alleles which have the potential for extensive crossreactivity and thus cover a large segment of the population.

### EXAMPLE 6

### Identification of Immunogenic Peptides

Using the B7-like-supermotifs identified above, sequences from potential antigenic sources including Hepatitis B Virus (HBV), Hepatitis C Virus (HCV), Human Papilloma Virus (HPV), Human Immunodeficiency Virus (HIV), MAGE2/3, and Plasmodium were analyzed for the presence of these motifs.

Sequences for the target antigens were obtained from the current GenBank data base. The identification of motifs was done using the “FINDPATTERNS” program (Devereux et al., Nucleic Acids Research 12:387-395 (1984)). A computer search was carried out for antigen proteins comprising the B7-like-supermotif.

Table 11 lists 244 peptides identified in this search. Accordingly, a preferred embodiment of the invention comprises a composition comprising a peptide of Table 11.

Other viral and tumor-related proteins can also be analyzed for the presence of these motifs. The amino acid sequence or the nucleotide sequence encoding products is obtained from the GenBank database in the cases of Prostate Specific antigen (PSA), p53 oncogene, Epstein Barr Nuclear Antigen-1 (EBNA-1), and c-erb2 oncogene (also called HER-2/neu).

In the cases of Hepatitis B Virus (HBV), Hepatitis C Virus (HCV), and Human Immunodeficiency Virus (HI) several strains/isolates exist and many sequences have been placed in GenBank.

For HBV, binding motifs are identified for the adr, adw and ayw types. In order to avoid replication of identical sequences, all of the adr motifs and only those motifs from adw and ayw that are not present in adr are added to the list of peptides.

In the case of HCV, a consensus sequence from residue 1 to residue 782 is derived from 9 viral isolates. Motifs are identified on those regions that have no or very little (one residue) variation between the 9 isolates. The sequences of residues 783 to 3010 from 5 viral isolates were also analyzed. Motifs common to all the isolates are identified and added to the peptide list.

Finally, a consensus sequence for HIV type 1 for North American viral isolates (10-12 viruses) was obtained from the Los Alamos National Laboratory database (May 1991 release) and analyzed in order to identify motifs that are constant throughout most viral isolates. Motifs that bear a small degree of variation (one residue, in 2 forms) were also added to the peptide list.

TABLE 11

| | | | | | | | | | | | | | MHC |
|---|---|---|---|---|---|---|---|---|---|---|---|---|---|
| Synthesis | Antigen | Mole | Size | Posi | Sequence | A1 | A2 | A3 | A1 | A24 | P1 | P2 | Alleles |
| CH-15 | CSP | | 9 | 293 | MPNDPNRNV | | | | | | + | | P1 |
| CH-15 | CSP | | 10 | 131 | NPDPNANPNV | | | | | | + | | P1 |
| CH-15 | CSP | | 10 | 318 | EFSDKHIKEY | + | | | | | | + | A01/P2 |
| CH-15 | HBV | ENV | 9 | 191 | IPQSLDSWW | | | | | | | + | P2 |
| CH-15 | HBV | ENV | 9 | 232 | CPGYRWMCL | | | | | | + | | P1 |
| CH-15 | HBV | ENV | 9 | 313 | IPIPSSWAF | | | | | | | + | P2 |
| CH-15 | HBV | POL | 9 | 365 | TPARVTGGV | | | | | | + | | P1 |
| CH-15 | HBV | ENV | 9 | 379 | LPIFFCLWV | | | | | | + | | P1 |
| CH-15 | HBV | POL | 9 | 404 | WPKFAVPNL | | | | | | + | | P1 |
| CH-15 | HBV | POL | 9 | 440 | HPAAMPHLL | | | | | | + | | P1 |
| CH-15 | HBV | POL | 9 | 541 | FPHCLAFSY | | | | | | | + | P2 |
| CH-15 | HBV | POL | 9 | 789 | DPSRGRLGL | | | | | | + | | P1 |
| CH-15 | HBV | POL | 10 | 19 | GPLEEELPRL | | | | | | + | | P1 |
| CH-15 | HBV | POL | 10 | 50 | IPWTHKVGNF | | | | | | | + | P2 |
| CH-15 | HBV | POL | 10 | 123 | LPLDKGIKPY | | | | | | | + | P2 |
| CH-15 | HBV | CORE | 10 | 134 | PPNAPILSTL | | | | | | + | | P1 |
| CH-15 | HBV | ENV | 10 | 173 | GPLLVLQAGF | | | | | | | + | P2 |
| CH-15 | HBV | ENV | 10 | 340 | VPFVQWFVGL | | | | | | + | | P1 |
| CH-15 | HBV | POL | 10 | 365 | TPARVTGGVF | | | | | | | + | P2 |
| CH-15 | HBV | ENV | 10 | 379 | LPIFFCLWVY | | | | | | | + | P2 |
| CH-15 | HBV | POL | 10 | 409 | VPNLQSLTNL | | | | | | + | | P1 |
| CH-15 | HBV | POL | 10 | 541 | FPHCLAFSYM | | | | | | + | | P1 |
| CH-15 | HCV | Core | 9 | 57 | QPRGRRQPI | | | | | | + | | P1 |
| CH-15 | HCV | Core | 9 | 78 | QPGYPWPLY | | | | | | | + | P2 |
| CH-15 | HCV | Core | 9 | 83 | WPLYGNEGL | | | | | | + | | P1 |
| CH-15 | HCV | Core | 9 | 99 | SPRGSRPSW | | | | | | | + | P2 |
| CH-15 | HCV | Core | 9 | 111 | DPRRRSRNL | | | | | | + | | P1 |
| CH-15 | HCV | Core | 9 | 168 | LPGCSFSIF | | | | | | | + | P2 |
| CH-15 | HCV | E1 | 9 | 339 | IPQAVVDMV | | | | | | + | | P1 |
| CH-15 | HCV | E2 | 9 | 600 | GPWLTPRCM | | | | | | + | | P1 |
| CH-15 | HCV | E2 | 9 | 622 | YPCTVNFTI | | | | | | + | | P1 |
| CH-15 | HCV | E2 | 9 | 681 | LPALSTGLI | | | | | | + | | P1 |
| CH-15 | HCV | NS3 | 9 | 1358 | HPNIEEVAL | | | | | | + | | P1 |
| CH-15 | HCV | NS3 | 9 | 1530 | TPAETTVRL | | | | | | + | | P1 |
| CH-15 | HCV | NS3 | 9 | 1598 | APPPSWDQM | | | | | | | + | P2 |
| CH-15 | HCV | NS3 | 9 | 1599 | PPPSWDQMW | | | | | | | + | P2 |
| CH-15 | HCV | NS3 | 9 | 1619 | GPTPLLYRL | | | | | | + | | P1 |
| CH-15 | HCV | NS4 | 9 | 1887 | SPGALVVGV | | | | | | + | | P1 |
| CH-15 | HCV | NS4 | 9 | 1906 | GPGEGAVQW | | | | | | | + | P2 |
| CH-15 | HCV | NS5 | 9 | 2159 | LPCEPEPDV | | | | | | + | | P1 |
| CH-15 | HCV | NS5 | 9 | 2162 | EPEPDVAVL | | | | | | + | | P1 |
| CH-15 | HCV | NS5 | 9 | 2396 | DPDLSDGSW | | | | | | | + | P2 |
| CH-15 | HCV | NS5 | 9 | 2512 | PPHSAKSKF | | | | | | | + | P2 |
| CH-15 | HCV | NS5 | 9 | 2615 | SPGQRVEFL | | | | | | + | | P1 |
| CH-15 | HCV | NS5 | 9 | 2771 | DPPQPEYDL | | | | | | + | | P1 |
| CH-15 | HCV | NS5 | 9 | 2774 | QPEYDLELI | | | | | | + | | P1 |
| CH-15 | HCV | NS5 | 9 | 2835 | APTLWARMI | | | | | | + | | P1 |
| CH-15 | HCV | Core | 10 | 37 | LPRRGPRLGV | | | | | | + | | P1 |
| CH-15 | HCV | Core | 10 | 142 | APLGGAARAL | | | | | | + | | P1 |
| CH-15 | HCV | Core | 10 | 168 | LPGCSFSIFL | | | | | | + | | P1 |
| CH-15 | HCV | E1 | 10 | 252 | IPTTTIRRHV | | | | | | + | | P1 |
| CH-15 | HCV | E1 | 10 | 308 | YPGHVSGHRM | | | | | | + | | P1 |
| CH-15 | HCV | E2 | 10 | 497 | VPASQVCGPV | | | | | | + | | P1 |
| CH-15 | HCV | E2 | 10 | 600 | GPWLTPRCMV | | | | | | + | | P1 |
| CH-15 | HCV | E2 | 10 | 622 | YPCTVNFTIF | | | | | | | + | P2 |
| CH-15 | HCV | E2 | 10 | 663 | SFLLLSTTEW | | | | | | | + | P2 |
| CH-15 | HCV | E2 | 10 | 793 | WPLLLLLLAL | | | | | | + | | P1 |
| CH-15 | HCV | NS3 | 10 | 1120 | TPCTCGSSDL | | | | | | + | | P1 |
| CH-15 | HCV | NS3 | 10 | 1239 | VPAAYAAQGY | + | | | | | | + | A01/P2 |
| CH-15 | HCV | NS3 | 10 | 1254 | NPSVAATLGF | | | | | | | + | P2 |
| CH-15 | HCV | NS3 | 10 | 1506 | RPSGMFDSSV | | | | | | + | | P1 |
| CH-15 | HCV | NS3 | 10 | 1547 | LPVCQDHLEF | | | | | | | + | P2 |
| CH-15 | HCV | NS3 | 10 | 1398 | APPPSWDQMW | | | | | | | + | P2 |
| CH-15 | HCV | NS3 | 10 | 1514 | KPTLHGPTPL | | | | | | + | | P1 |
| CH-15 | HCV | NS3 | 10 | 1521 | TPLLYRLGAV | | | | | | + | | P1 |
| CH-15 | HCV | NS4 | 10 | 1730 | LPGNPAIASL | | | | | | + | | P1 |
| CH-15 | HCV | NS4 | 10 | 1733 | NPAIASLMAF | | | | | | | + | P2 |
| CH-15 | HCV | NS4 | 10 | 1532 | LPAILSPGAL | | | | | | + | | P1 |
| CH-15 | HCV | NS4 | 10 | 1337 | SPGALVVGVV | | | | | | + | | P1 |
| CH-15 | HCV | NS4 | 10 | 1906 | GPGEGAVQWM | | | | | | + | | P1 |
| CH-15 | HCV | NS4 | 10 | 1934 | VPESDAAARV | | | | | | + | | P1 |
| CH-15 | HCV | NS5 | 10 | 1164 | EPDVAVLTSM | | | | | | + | | P1 |
| CH-15 | HCV | NS5 | 10 | 2515 | SPGQRVEFLV | | | | | | + | | P1 |
| CH-15 | HCV | NS5 | 10 | 2758 | PPGDPPQPEY | | | | | | | + | P2 |
| CH-15 | HCV | NS5 | 10 | 2772 | PPQPEYDLEL | | | | | | + | | P1 |
| CH-15 | HCV | NS5 | 10 | 2322 | TPVNSWLGNI | | | | | | + | | P1 |
| CH-15 | HCV | NS5 | 10 | 2335 | APTLWARMIL | | | | | | + | | P1 |
| CH-15 | HIV | VPR | 9 | 34 | FPRIWLHJL | | | | | | + | | P1 |
| CH-15 | HIV | POL | 9 | 37 | SPTRRELQV | | | | | | + | | P1 |
| CH-15 | HIV | NEF | 9 | 34 | FPVRPQVPL | | | | | | + | | P1 |
| CH-15 | HIV | NEF | 9 | 37 | RPQVPLRPM | | | | | | + | | P1 |
| CH-15 | HIV | VIF | 9 | 99 | DPDLADQLI | | | | | | + | | P1 |
| CH-15 | HIV | POL | 9 | 110 | LPGRWKPKM | | | | | | + | | P1 |
| CH-15 | HIV | ENV | 9 | 123 | KPCVKLTPL | | | | | | + | | P1 |
| CH-15 | HIV | GAG | 9 | 153 | SPRTLNAWV | | | | | | + | | P1 |
| CH-15 | HIV | VIF | 9 | 161 | PPLPSVJKL | | | | | | + | | P1 |
| CH-15 | HIV | POL | 9 | 171 | FPISPIETV | | | | | | + | | P1 |
| CH-15 | HIV | POL | 9 | 179 | VPVKLKPGM | | | | | | + | | P1 |
| CH-15 | HIV | POL | 9 | 184 | KPGMDGPKV | | | | | | | + | P2 |
| CH-15 | HIV | GAG | 9 | 185 | TPQDLNTML | | | | | | + | | P1 |
| CH-15 | HIV | POL | 9 | 189 | GPKVKQWPL | | | | | | | + | P2 |
| CH-15 | HIV | GAG | 9 | 258 | NPPIPVGEI | | | | | | + | | P1 |
| CH-15 | HIV | GAG | 9 | 259 | PPIPVGEIY | | | | | | | + | P2 |
| CH-15 | HIV | GAG | 9 | 293 | GPKEPFRDY | | | | | | | + | P2 |
| CH-15 | HIV | POL | 9 | 327 | SPAIFQSSM | | | | | | + | | P1 |
| CH-15 | HIV | GAG | 9 | 343 | GPAATLEEM | | | | | | + | | P1 |
| CH-15 | HIV | POL | 9 | 346 | NPDIVIYQY | + | | | | | | + | A01/P2 |
| CH-15 | HIV | GAG | 9 | 360 | GPGHKARVL | | | | | | + | | P1 |
| CH-15 | HIV | POL | 9 | 395 | EPPFLWMGY | | | | | | | + | P2 |
| CH-15 | HIV | ENV | 9 | 404 | DPEIVMHSF | | | | | | | + | P2 |
| CH-15 | HIV | POL | 9 | 417 | LPEKDSWTV | | | | | | | + | P2 |
| CH-15 | HIV | GAG | 9 | 507 | YPLASLRSL | | | | | | + | | P1 |
| CH-15 | HIV | ENV | 9 | 547 | APTKAKRRV | | | | | | + | | P1 |
| CH-15 | HIV | POL | 9 | 590 | TPPLVKLWY | | | | | | | + | P2 |
| CH-15 | HIV | POL | 9 | 603 | EPIVGAETF | | | | | | | + | P2 |
| CH-15 | HIV | POL | 9 | 680 | QPDKSESEL | | | | | | + | | P1 |
| CH-15 | HIV | POL | 9 | 759 | LPPVVAKEI | | | | | | + | | P1 |
| CH-15 | HIV | POL | 9 | 760 | PPVVAKEIV | | | | | | + | | P1 |
| CH-15 | HIV | POL | 9 | 991 | VPRRKAKII | | | | | | + | | P1 |
| CH-15 | HIV | TAT | 10 | 2 | EPVDPRLEPW | | | | | | | + | P2 |
| CH-15 | HIV | POL | 10 | 37 | SPTRRELQVW | | | | | | | + | P2 |
| CH-15 | HIV | POL | 10 | 110 | LPGRWKPKMI | | | | | | + | | P1 |
| CH-15 | HIV | POL | 10 | 152 | TPVNIIGRNL | | | | | | + | | P1 |
| CH-15 | HIV | VIF | 10 | 160 | KPPLPSVJKL | | | | | | + | | P1 |
| CH-15 | HIV | POL | 10 | 174 | SPIETVPVKL | | | | | | + | | P1 |
| CH-15 | HIV | POL | 10 | 222 | GPENPYNTPV | | | | | | + | | P1 |
| CH-15 | HIV | POL | 10 | 225 | NPYNTPVFAI | | | | | | + | | P1 |
| CH-15 | HIV | GAG | 10 | 258 | NPPIPVGEIY | | | | | | | + | P2 |
| CH-15 | HIV | GAG | 10 | 261 | IPVGEIYKRW | | | | | | | + | P2 |
| CH-15 | HIV | POL | 10 | 289 | VPLDKDFRKY | | | | | | | + | P2 |
| CH-15 | HIV | GAG | 10 | 293 | GPKEPFRDYV | | | | | | + | | P1 |
| CH-15 | HIV | GAG | 10 | 296 | EPFRDYVDRF | | | | | | | + | P2 |
| CH-15 | HIV | POL | 10 | 310 | TPGIRYQYNV | | | | | | + | | P1 |
| CH-15 | HIV | POL | 10 | 340 | EPFRKQNPDI | | | | | | + | | P1 |
| CH-15 | HIV | GAG | 10 | 343 | GPAATLEEMM | | | | | | + | | P1 |
| CH-15 | HIV | POL | 10 | 346 | NPDIVIYQYM | | | | | | + | | P1 |
| CH-15 | HIV | POL | 10 | 396 | PPFLWMGYEL | | | | | | + | | P1 |
| CH-15 | HIV | POL | 10 | 406 | HPDKWTVQPI | | | | | | + | | P1 |
| CH-15 | HIV | GAG | 10 | 473 | EPTAPPEESF | | | | | | | + | P2 |
| CH-15 | HIV | GAG | 10 | 507 | YPLASLRSLF | | | | | | | + | P2 |
| CH-15 | HIV | ENV | 10 | 547 | APTKAKRRVV | | | | | | + | | P1 |
| CH-15 | HIV | POL | 10 | 591 | PPLVKLWYQL | | | | | | + | | P1 |
| CH-15 | HIV | POL | 10 | 603 | EPIVGAETFY | | | | | | | + | P2 |
| CH-15 | HIV | POL | 10 | 680 | QPDKSESELV | | | | | | + | | P1 |
| CH-15 | HIV | POL | 10 | 759 | LPPVVAKEIV | | | | | | + | | P1 |
| CH-15 | HIV | POL | 10 | 872 | IPYNPQSQGV | | | | | | + | | P1 |
| CH-15 | HIV | POL | 10 | 963 | DPLWKGPAKL | | | | | | + | | P1 |
| CH-15 | HPV16 | E7 | 9 | 5 | TPTLHEYML | | | | | | + | | P1 |
| CH-15 | HPV16 | E6 | 9 | 11 | DPQERPRKL | | | | | | + | | P1 |
| CH-15 | HPV16 | E7 | 9 | 46 | EPDRAHYNI | | | | | | + | | P1 |
| CH-15 | HPV16 | E6 | 9 | 118 | CPEEKQRHL | | | | | | + | | P1 |
| CH-15 | HPV16 | E7 | 10 | 46 | EPDRAHYNIV | | | | | | + | | P1 |
| CH-15 | HPV16 | E6 | 10 | 65 | NPYAVCDKCL | | | | | | + | | P1 |
| CH-15 | HPV18 | E7 | 9 | 3 | GPKATLQDI | | | | | | + | | P1 |
| CH-15 | HPV18 | E6 | 9 | 6 | DPTRRPYKL | | | | | | + | | P1 |
| CH-15 | HPV18 | E6 | 9 | 110 | KPLNPAEKL | | | | | | + | | P1 |
| CH-15 | HPV18 | E6 | 9 | 113 | NPAEKLRHL | | | | | | + | | P1 |
| CH-15 | HPV18 | E7 | 10 | 3 | GPKATLQDIV | | | | | | + | | P1 |
| CH-15 | HPV18 | E7 | 10 | 16 | EPQNEIPVDL | | | | | | + | | P1 |
| CH-15 | HPV18 | E7 | 10 | 55 | EFQRHTMLCM | | | | | | + | | P1 |
| CH-15 | HPV18 | E6 | 10 | 60 | IPHAACHKCI | | | | | | + | | P1 |
| CH-15 | LSA1 | | 9 | 1663 | LPSENERGY | + | | | | | | + | A01/P2 |
| CH-15 | LSA1 | | 9 | 1786 | KPIVQYDNF | | | | | | | + | P2 |
| CH-15 | LSA1 | | 10 | 1663 | LPSENERGYY | + | | | | | | + | A01/P2 |
| CH-15 | MAGE2 | | 9 | 170 | VPISHLYIL | | | | | | + | | P1 |
| CH-15 | MAGE2 | | 9 | 196 | MPKTGLLII | | | | | | | + | P2 |
| CH-15 | MAGE2 | | 9 | 265 | DPACYEFLW | | | | | | | + | P2 |
| CH-15 | MAGE2 | | 9 | 296 | EPHISYPPL | | | | | | + | | P1 |
| CH-15 | MAGE2 | | 9 | 301 | YPPLHERAL | | | | | | + | | P1 |
| CH-15 | MAGE2 | | 10 | 170 | VPISHLYILV | | | | | | + | | P1 |
| CH-15 | MAGE2 | | 10 | 196 | MPKTGLLIIV | | | | | | + | | P1 |
| CH-15 | MAGE2 | | 10 | 241 | HPRKLLMQDL | | | | | | | + | P2 |
| CH-15 | MAGE2 | | 10 | 274 | GPRALIETSY | | | | | | | + | P2 |
| CH-15 | MAGE2/3 | | 9 | 128 | EPVTKAEML | | | | | | + | | P1 |
| CH-15 | MAGE2/3 | | 9 | 261 | VPGSDPACY | | | | | | | + | P2 |
| CH-15 | MAGE2/3 | | 10 | 216 | APEEKIWEEL | | | | | | + | | P1 |
| CH-15 | MAGE3 | | 9 | 71 | LPTTMNYPL | | | | | | + | | P1 |
| CH-15 | MAGE3 | | 9 | 170 | DPIGHLYIF | | | | | | | + | P2 |
| CH-15 | MAGE3 | | 9 | 196 | MPKAGLLII | | | | | | + | | P1 |
| CH-15 | MAGE3 | | 9 | 296 | GPHISYPPL | | | | | | + | | P1 |
| CH-15 | MAGE3 | | 9 | 302 | YPPLHEWVL | | | | | | + | | P1 |
| CH-15 | MAGE3 | | 10 | 71 | LPTTMNYPLW | | | | | | | + | P2 |
| CH-15 | MAGE3 | | 10 | 196 | MPKAGLLIIV | | | | | | + | | P1 |
| CH-15 | MAGE3 | | 10 | 241 | DPKKLLTQHF | | | | | | | + | P2 |
| CH-15 | MAGE3 | | 10 | 274 | GPRALVETSY | | | | | | | + | P2 |
| CH-15 | SSP2 | | 9 | 164 | IPDSIQDSL | | | | | | + | | P1 |
| CH-15 | SSP2 | | 9 | 206 | HPSDGKCNL | | | | | | + | | P1 |
| CH-15 | SSP2 | | 9 | 228 | GPFMKAVCV | | | | | | + | | P1 |
| CH-15 | SSP2 | | 9 | 287 | LPKREPLDV | | | | | | + | | P1 |
| CH-15 | SSP2 | | 9 | 305 | RPRGDNFAV | | | | | | + | | P1 |
| CH-15 | SSP2 | | 9 | 364 | PPNPPNPDI | | | | | | + | | P1 |
| CH-15 | SSP2 | | 9 | 379 | IPEDSEKEV | | | | | | + | | P1 |
| CH-15 | SSP2 | | 9 | 544 | EPAPFDETL | | | | | | + | | P1 |
| CH-15 | SSP2 | | 9 | 303 | QPRPRGDNF | | | | | | | + | P2 |
| CH-15 | SSP2 | | 9 | 419 | LPNDKSDRY | | | | | | | + | P2 |
| CH-15 | SSP2 | | 10 | 363 | NPPNPPNPDI | | | | | | + | | P1 |
| CH-15 | SSP2 | | 10 | 419 | LPNDKSDRYI | | | | | | + | | P1 |
| CH-15 | SSP2 | | 10 | 428 | IPYSPLSPKV | | | | | | + | | P1 |
| CH-15 | SSP2 | | 10 | 206 | HPSDGKCNLY | + | | | | | | + | A01/P2 |
| CH-15 | SSP2 | | 10 | 394 | NPEDDREENF | | | | | | | + | P2 |
| CH-15 | SSP2 | | 10 | 539 | TPYAGEPAPF | | | | | | | + | P2 |

| X | Source | Mol. | Pos. | Cytel # | Sequence | AA | Motif |
|---|---|---|---|---|---|---|---|
| 1 | HBV | ENV | 14 | 16.006 | FPDNQLDPA | 9 | P2A |
| 2 | HBV | NUC | 129 | 16.007 | PPAYRPPNA | 9 | P2A |
| 3 | HBV | POL | 640 | 16.008 | YPALMPLYA | 9 | P2A |
| 4 | HBV | X | 58 | 16.009 | LPVCAFSSA | 9 | P2A |
| 5 | HCV | | 142 | 16.010 | APLGGAARA | 9 | P2A |
| 6 | HCV | | 2806 | 16.011 | DPTTPLARA | 9 | P2A |
| 7 | HCV | | 1582 | 16.012 | FPYLVAYQA | 9 | P2A |
| 8 | HCV | | 1882 | 16.013 | LPAILSPGA | 9 | P2A |
| 9 | HCV | | 1783 | 16.014 | NPAIASLMA | 9 | P2A |
| 10 | HCV | | 2897 | 16.015 | SPGEINRVA | 9 | P2A |
| 11 | HCV | | 2551 | 16.016 | TPIDTTIMA | 9 | P2A |
| 12 | HCV | | 1621 | 16.017 | TPLLYRLGA | 9 | P2A |
| 13 | HCV | | 242 | 16.018 | TPTLAARNA | 9 | P2A |
| 14 | HCV | | 793 | 16.019 | WPLLLLLLA | 9 | P2A |
| 15 | HIV | NEF | 38 | 16.020 | EPAADGVGA | 9 | P2A |
| 16 | HIV | POL | 225 | 16.021 | NPYNTPVFA | 9 | P2A |
| 17 | MAGE2 | | 60 | 16.022 | SPPHSPQGA | 9 | P2A |
| 18 | MAGE3 | | 30 | 16.023 | APATEEQEA | 9 | P2A |
| 19 | MAGE3 | | 60 | 16.024 | DPPQSPQGA | 9 | P2A |
| 20 | PAP | | 4 | 16.032 | APLLLARAA | 9 | P2A |
| 21 | Plasmodium | TRAP | 522 | 16.175 | VPGAATPYA | 9 | P2A |
| 22 | PSA | | 52 | 16.176 | HPQWVLTAA | 9 | P2A |
| 23 | HBV | ENV | 313 | 16.177 | IPIPSSWAFA | 10 | P2A |
| 24 | HBV | NUC | 49 | 16.178 | SPHHTALRQA | 10 | P2A |
| 25 | HBV | NUC | 128 | 16.179 | TPPAYRPPNA | 10 | P2A |
| 26 | HBV | POL | 633 | 16.180 | APFTQCGYPA | 10 | P2A |
| 27 | HBV | POL | 712 | 16.181 | LPIHTAELLA | 10 | P2A |
| 28 | HBV | X | 67 | 16.182 | GPCALRFTSA | 10 | P2A |
| 29 | HCV | | 2181 | 16.183 | DPSHITAETA | 10 | P2A |
| 30 | HCV | | 2806 | 16.184 | DPTTPLARAA | 10 | P2A |
| 31 | HCV | | 339 | 16.185 | IPQAVVDMVA | 10 | P2A |
| 32 | HCV | | 2159 | 16.186 | LPCEPEPDVA | 10 | P2A |
| 33 | HCV | | 674 | 16.187 | LPCSFTTLPA | 10 | P2A |
| 34 | HCV | | 2567 | 16.188 | QPEKGGRKPA | 10 | P2A |
| 35 | HCV | | 1356 | 16.189 | VPHPNIEEVA | 10 | P2A |
| 36 | HIV | GAG | 360 | 16.190 | GPGHKARVLA | 10 | P2A |
| 37 | HIV | GAG | 332 | 16.191 | NPDCKTILKA | 10 | P2A |
| 38 | HIV | GAG | 170 | 16.192 | SPEVIPMFSA | 10 | P2A |
| 39 | HIV | POL | 820 | 16.195 | IPAETGQETA | 10 | P2A |
| 40 | HIV | POL | 320 | 16.196 | LPQGWKGSPA | 10 | P2A |
| 41 | HIV | POL | 760 | 16.197 | PPvVAKEIVA | 10 | P2A |
| 42 | MAGE2 | | 30 | 16.198 | APATEEQQTA | 10 | P2A |
| 43 | MAGE2/3 | | 98 | 16.199 | FPDLESEFQA | 10 | P2A |
| 44 | MAGE3 | | 30 | 16.200 | APATEEQEAA | 10 | P2A |
| 45 | MAGE3 | | 170 | 16.201 | DPIGHLYIFA | 10 | P2A |
| 46 | PAP | | 348 | 16.202 | SPSCPLERFA | 10 | P2A |
| 47 | Plasmodium | CSP | 327 | 16.218 | DPNRNVDENA | 10 | P2A |
| 48 | Plasmodium | EXP-1 | 116 | 16.243 | DPADNANPDA | 10 | P2A |
| 49 | Plasmodium | EXP-1 | 132 | 16.244 | EPNADPQVTA | 10 | P2A |
| 50 | Plasmodium | LSA1 | 1728 | 16.307 | KPEQKEDKSA | 10 | P2A |
| 51 | Plasmodium | TRAP | 303 | 16.342 | QPRPRGDNFA | 10 | P2A |
| 52 | PSA | | 141 | 16.343 | EPALGTTCYA | 10 | P2A |

EXAMPLE 7

### Ex vivo Induction of Cytotoxic T Lymphocytes (CTM)

Peripheral blood mononuclear cells (PBMC) are isolated from an HLA-typed patient by either venipuncture or apheresis (depending upon the initial amount of CTLp required), and purified by gradient centrifugation using Ficoll-Paque (Pharmacia). Typically, one can obtain one million PBMC for every ml of peripheral blood, or alternatively, a typical apheresis procedure can yield up to a total of 1-10×10¹⁰ PBMC.

The isolated and purified PBMC are co-cultured with an appropriate number of antigen presenting cell (APC), previously incubated (“pulsed”) with an appropriate amount of synthetic peptide (containing the HLA binding motif and the sequence of the antigen in question). PBMC are usually incubated at 1-2×10⁶ cells/ml in culture medium such as RPMI-1640 (with autologous serum or plasma) or the serum-free medium AIM-V (Gibco).

APC are usually used at concentrations ranging from 1×10⁴ to 2×10⁵ cells/ml, depending on the type of cell used. Possible sources of APC include: 1) autologous dendritic cells (DC), which are isolated from PBMC and purified as described (Inaba, et al., J. Exp. Med. 166:182 (1987)); and 2) mutant and genetically engineered mammalian cells that express “empty” HLA molecules (which are syngeneic [genetically identical] to the patient's allelic HLA form), such as the, mouse RMA-S cell line or the human T2 cell line. APC containing empty HLA molecules are known to be potent inducers of CTL responses, possibly because the peptide can associate more readily with empty MHC molecules than with MHC molecules which are occupied by other peptides (DeBruijn, et al., Eur. J. Immunol. 21:2963-2970 (1991)).

In those cases when the APC used are not autologous, the cells will have to be gamma irradiated with an appropriate dose (using, e.g., radioactive cesium or cobalt) to prevent their proliferation both ex vivo, and when the cells are re-introduced into the patients.

The mixture cultures, containing PBMC, APC and peptide are kept in an appropriate culture vessel such as plastic T-flasks, gas-permeable plastic bags, or roller bottles, at 37° centigrade in a humid air/CO₂ incubator. After the activation phase of the culture, which usually occurs during the first 3-5 days, the resulting effector CTL can be further expanded, by the addition of recombinant DNA-derived growth factors such as interleukin-2 (IL-2), interleukin-4 (IL-4), or interleukin-7 (IL-7) to the cultures. An expansion culture can be kept for an additional 5 to 12 days, depending on the numbers of effector CTL required for a particular patient. In addition, expansion cultures may be performed using hollow fiber artificial capillary systems (Cellco), where larger numbers of cells (up to 1×10¹¹) can be maintained.

Before the cells are infused into the patient, they are tested for activity, viability, toxicity and sterility. The cytotoxic activity of the resulting CTL can be determined by a standard ^{5I}Cr-release assay (Biddison, W. E. 1991, Current Protocols in Immunology, p7,17.1-7.17.5, Ed. J. Coligan et al., J. Wiley and Sons, New York), using target cells that express the appropriate HLA molecule, in the presence and absence of the immunogenic peptide. Viability is determined by the exclusion of trypan blue dye by live cells. Cells are tested for the presence of endotoxin by conventional techniques. Finally, the presence of bacterial or fungal contamination is determined by appropriate microbiological methods (chocolate agar, etc.). Once the cells pass all quality control and safety tests, they are washed and placed in the appropriate infusion solution (Ringer/glucose lactate) and infused intravenously into the patient.

### EXAMPLE 8

### Binding of Peptides to B7-Like Supermotif HLA Alleles

Peptides bearing the B7-like supermotif were tested for binding to purified HLA molecules of some of the alleles sharing the B7-like specificity. The binding assay was performed as described in Example 2. Table 12 shows the binding to HLA-B*0701, B*3501, B*3502, B*3503, and B*5401 of a set of peptides reported in the literature to be restricted or naturally bound to various HLA-B alleles.

Table 13 shows the binding of a set of 124 9-mer and 124 10-mer B7-like supermotif bearing peptides of various viral and bacterial origin to HLA-B*0701, B*3501, B*5301, and B*5401. In general, immunogenicity is correlated with binding affinity in that peptides which bind MHC with affinities of 500 nM or less show greater immunogenicity.

As shown in Tables 12 and 13, there are peptides which are capable of binding to more than one allele, demonstrating that molecules of the defined B7-like supermotif family are indeed capable of binding overlapping sets of peptides. To date, approximately 10 peptides capable of over 25% (at minimum) population coverage, as defined through its binding to any B7-like allele(s), have been identified (Table 14). HBV, HIV, HCV, Mage 2, Mage 3, and P. falciparum are each represented by at least one cross-reactive binder.

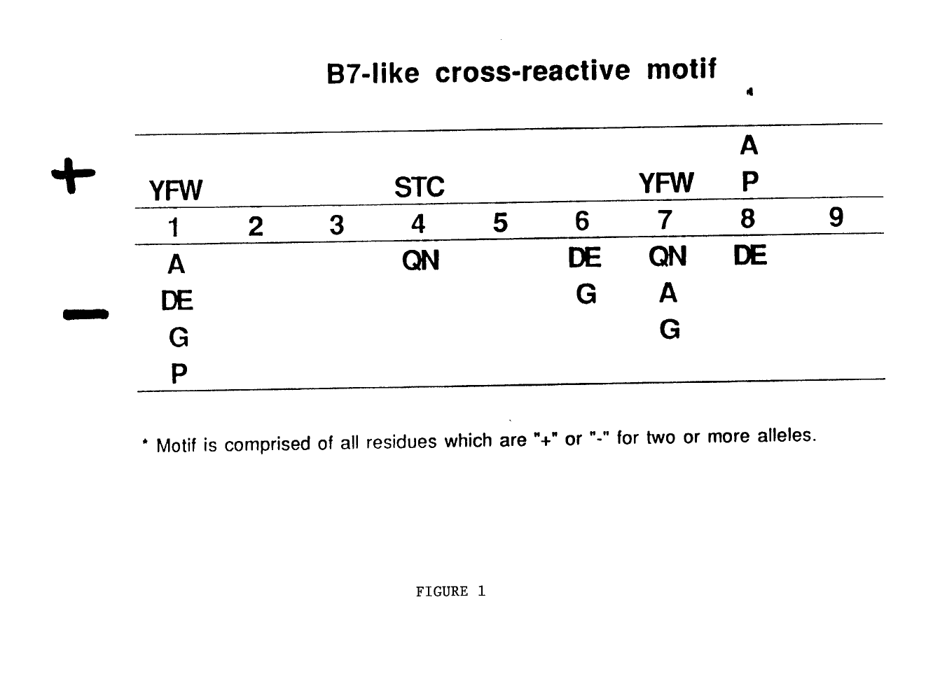

The basis for the observed cross-reactivity was examined by first establishing for four alleles, B*0701, B*3501, B*5301, and B*5401, their individual secondary anchor motifs (FIG. 1). From the individual motifs, a B7-like cross reactive motif is comprised of all residues which are positive secondary anchors for at least 2 of the four alleles examined. In its negative aspect, the motif excludes peptides bearing residues at certain positions which are detrimental influences on binding for at least 2 of the four alleles examined. As shown in Table 15, the B7-like cross-reactive supermotif allows the improves prediction of peptides which will be capable of binding to 2 or more alleles of the B7-like superfamily.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

TABLE 12
Binding of B7-like supermotif containing peptides to B7-like supertype HLA alleles

| | | RESTRICTION | | BINDING CAPACITY (IC50 nM) | | | | |
|---|---|---|---|---|---|---|---|---|
| SEQUENCE | SOURCE | (or ORIGIN) | REFERENCE | B′0701 | B′3501 | B′3502 | B′3503 | B′5401 |
| YPAEITLTW | B′5301 self peptide | B′5301 | 38 | 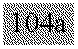 | 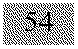 | 1160 | 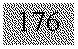 | 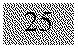 |
| MPLETQLAI | P. talciparum SHEBA 77-85 | B′5101, B′5301 | 38 | 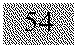 | 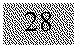 | 1146 | 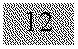 | 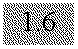 |
| LPSDFFPSV | HBc 19-27 | | | 1323 | 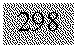 | — | 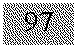 | 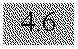 |
| XPSDXAAEA | B′5401 Nat. Processed | (B′5401) | | 11714 | 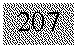 | 13364 | 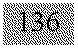 | 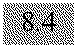 |
| LPFDFTPGY | B′3501 nat. proc. | (B′3501) | 83 | -b) | 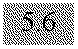 | 1307 | 6286 | 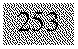 |
| APRTVALTA | B′0701 Nat. Processed | (B′0701) | 59 | 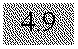 | — | — | — | 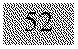 |
| LPGPKFLQY | B′3501 nat. proc. | (B′3501) | 83 | — | 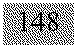 | — | — | — |
| DPKVKQWPL | HIV pol 185-193 | B′0801 | 72 | 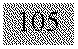 | 5636 | — | 1128 | 17813 |
| MPNDPNRNV | P. falciparum cap 300-308 | B′5101, B′5301 | 38 | 3417 | — | — | — | 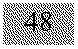 |
| APRTLVYLL | A′0201 sig seq 5-13 analog | (B′0701) | 59 | 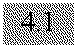 | — | — | — | — |
| APRTVALTAL | B′0701 Nat. Processed | (B′0701) | 59 | 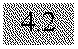 | — | — | — | 17273 |
| APRASRPSL | B′0701 Nat. Processed | (B′0701) | 59 | 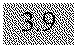 | — | — | — | — |
| YPFQPPKV | B′5401 Nat. Processed | (B′5401) | | — | — | — | 14667 | 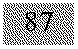 |
| KPIVQYDNF | P. falciperum isa 1786-1794 | B′5301 | 38 | 27333 | — | — | — | — |
| TPYDINQML | HIV-2 | B′5301 | 38 | 2733 | 17714 | — | 1158 | 15833 |
| DPYEVSYRI | B′5401 Nat. Processed | (B′5401) | | — | — | — | — | — |

a) Shaded areas highlight binding capacity
b) A dash indicates an IC50 >30000 nM

TABLE 13
Binding of peptides to B7-like supermotif alleles

| | | | | | | | | | | | | | B*0701 | B*3501 | B*5301 | B*5401 | Alleles |
|---|---|---|---|---|---|---|---|---|---|---|---|---|---|---|---|---|---|
| PEPTIDE | AA | P1 | P2 | P3 | P4 | P5 | P6 | P7 | P8 | P9 | P10 | SOURCE | (nM) | (nM) | (nM) | (nM) | bound |
| 15.066 | 9 | F | P | V | R | P | Q | V | P | L | | HIV NEF 84 | 7.1 | 22 | 192 | 44 | 4 |
| 15.032 | 9 | I | P | I | P | S | S | W | A | F | | HBV ENV 313 | 60 | 78 | 35 | 4000 | 3 |
| 15.037 | 9 | F | P | H | C | L | A | F | S | Y | | HBV POL 541 | 3375 | 75 | 18 | 400 | 3 |
| 15.044 | 9 | L | P | G | C | S | F | S | I | F | | HCV Core 168 | 61 | 113 | 122 | 8000 | 3 |
| 15.107 | 9 | V | P | I | S | H | L | Y | I | L | | MAGE2 170 | 22 | 384 | 396 | 3525 | 3 |
| 15.140 | 9 | M | P | K | A | G | L | L | I | I | | MAGE3 196 | 320 | — | 92 | 112 | 3 |
| 16.009 | 9 | L | P | V | C | A | F | S | S | A | | HBV X 58 | 348 | 533 | — | 2.0 | 2 |
| 15.047 | 9 | Y | P | C | T | V | N | F | T | I | | HCV E2 622 | 10800 | 966 | 102 | 89 | 2 |
| 16.012 | 9 | F | P | Y | L | V | A | Y | Q | A | | HCV 1582 | 18000 | 182 | 1706 | 12 | 2 |
| 15.064 | 9 | F | P | R | I | W | L | H | J | L | | HIV VPR 34 | 5.4 | 10286 | 16909 | 226 | 2 |
| 15.073 | 9 | F | P | I | S | P | I | E | T | V | | HIV POL 171 | 3484 | 1051 | 251 | 9.8 | 2 |
| 15.134 | 9 | L | P | T | T | M | N | Y | P | L | | MAGE3 71 | 71 | 46 | 802 | 3152 | 2 |
| 16.032 | 9 | A | P | L | L | L | A | R | A | A | | PAP 4 | 257 | — | — | 2.6 | 2 |
| 16.176 | 9 | H | P | Q | W | V | L | T | A | A | | PSA 52 | 225 | 1532 | — | 1.1 | 2 |
| 15.030 | 9 | I | P | Q | S | L | D | S | W | W | | HBV ENV 191 | — | — | 64 | — | 1 |
| 15.033 | 9 | T | P | A | R | V | T | G | G | V | | HBV POL 365 | 466 | — | — | 18909 | 1 |
| 15.034 | 9 | L | P | I | F | F | C | L | W | V | | HBV ENV 379 | — | — | 2345 | 55 | 1 |
| 15.036 | 9 | H | P | A | A | M | P | H | L | L | | HBV POL 440 | 58 | 1618 | 580 | 6118 | 1 |
| 15.038 | 9 | D | P | S | R | G | R | L | G | L | | HBV POL 789 | 45 | — | — | — | 1 |
| 16.006 | 9 | F | P | D | H | Q | L | D | P | A | | HBV ENV 14 | — | 8000 | — | 13 | 1 |
| 16.008 | 9 | Y | P | A | L | M | P | L | Y | A | | HCV POL 640 | 524 | 1134 | 2583 | 080 | 1 |
| 15.039 | 9 | Q | P | R | G | R | R | Q | P | I | | HCV Core 57 | 24 | — | — | — | 1 |
| 15.042 | 9 | S | P | R | G | S | R | P | S | W | | HCV Core 99 | 14 | — | — | — | 1 |
| 15.043 | 9 | D | P | R | R | R | S | R | N | L | | HCV Core 111 | 318 | — | — | — | 1 |
| 15.048 | 9 | L | P | A | L | S | T | G | L | I | | HCV E2 681 | 153 | — | 1505 | 20800 | 1 |
| 15.049 | 9 | H | P | N | I | E | E | V | A | L | | HCV NS3 1358 | 1500 | 227 | 14308 | 5333 | 1 |
| 15.054 | 9 | S | P | G | A | L | V | V | G | V | | HCV NS4 1887 | — | — | 81 | — | 1 |
| 15.060 | 9 | S | P | G | Q | R | V | E | F | L | | HCV NS5 2615 | 44 | — | — | — | 1 |
| 15.063 | 9 | A | P | T | L | W | A | R | M | I | | HCV NS5 2835 | 338 | — | — | — | 1 |
| 16.010 | 9 | A | P | L | G | G | A | A | R | A | | HCV 142 | 1385 | — | — | 330 | 1 |
| 16.013 | 9 | L | P | A | I | L | S | P | G | A | | HCV 1882 | — | — | — | 11 | 1 |
| 16.014 | 9 | N | P | A | I | A | S | L | M | A | | HCV 1783 | 5143 | — | — | 263 | 1 |
| 16.016 | 9 | T | P | I | D | T | T | I | M | A | | HCV 2551 | 10800 | 14400 | — | 24 | 1 |
| 16.017 | 9 | T | P | I | D | T | T | I | M | A | | HCV 1621 | 655 | — | — | 45 | 1 |
| 16.019 | 9 | W | P | L | L | L | L | L | L | A | | HCV 793 | 10800 | — | 12400 | 270 | 1 |
| 15.065 | 9 | S | P | T | R | R | E | L | Q | V | | HIV POL 37 | 257 | — | — | — | 1 |
| 15.067 | 9 | R | P | Q | V | P | L | R | P | M | | HIV NEF 87 | 3.3 | 5760 | — | — | 1 |
| 15.070 | 9 | K | P | C | V | K | L | T | P | L | | HIV ENV 123 | 13 | — | — | — | 1 |
| 15.071 | 9 | S | P | R | T | L | N | A | W | V | | HIV GAG 153 | 9.8 | — | — | 20800 | 1 |
| 15.077 | 9 | G | P | K | V | K | Q | W | P | L | | HIV POL 189 | 372 | — | — | — | 1 |
| 15.081 | 9 | S | P | A | I | F | Q | S | S | M | | HIV POL 327 | 13 | 1920 | — | 8000 | 1 |
| 15.083 | 9 | N | P | D | I | V | I | Y | Q | Y | | HIV POL 346 | — | 343 | — | — | 1 |
| 15.084 | 9 | G | P | G | H | K | A | R | V | L | | HIV GAG 36O | 189 | — | — | — | 1 |
| 15.088 | 9 | Y | P | L | A | S | L | R | S | L | | HIV GAG 507 | 5.5 | 847 | 11625 | 1944 | 1 |
| 15.095 | 9 | V | P | R | R | K | A | K | I | I | | HIV POL 991 | 11 | — | — | — | 1 |
| 16.021 | 9 | N | P | Y | N | T | P | V | F | A | | HIV POL 225 | — | — | — | 105 | 1 |
| 15.096 | 9 | T | P | T | L | H | E | Y | M | L | | HPV16 E7 5 | 51 | — | — | — | 1 |
| 15.104 | 9 | K | P | L | N | P | A | E | K | L | | HPV18E6 110 | 154 | — | — | — | 1 |
| 15.108 | 9 | M | P | K | T | G | L | L | I | I | | MAGE2 196 | 2769 | — | 172 | 597 | 1 |
| 15.113 | 9 | D | P | A | C | Y | E | F | L | W | | MAGE2 265 | — | — | 115 | — | 1 |
| 15.117 | 9 | E | P | H | I | S | Y | P | P | L | | MAGE2 296 | 50 | 8000 | — | — | 1 |
| 15.119 | 9 | Y | P | P | L | H | E | R | A | L | | MAGE2 301 | 20 | — | — | 5474 | 1 |
| 15.156 | 9 | G | P | H | I | S | Y | P | P | L | | MAGE3 296 | 6.2 | — | — | — | 1 |
| 15.175 | 9 | H | P | S | D | G | K | C | N | L | | SSP2 206 | 245 | — | 6414 | — | 1 |
| 15.178 | 9 | R | P | R | G | D | N | F | A | V | | SSP2 305 | 11 | — | — | 3506 | 1 |
| 15.182 | 9 | Q | P | R | P | R | G | D | N | F | | SSP2 303 | 331 | — | — | — | 1 |
| 15.031 | 9 | C | P | G | Y | R | W | M | C | L | | HBV ENV 232 | 806 | — | — | — | 0 |
| 15.035 | 9 | W | P | K | F | A | V | P | N | L | | HBV POL 404 | 1009 | — | — | 7172 | 0 |
| 16.007 | 9 | P | P | A | Y | R | P | P | N | A | | HBV NUC 129 | — | — | — | — | 0 |
| 15.040 | 9 | Q | P | G | Y | P | W | P | L | Y | | HCV Core 78 | — | 6545 | — | — | 0 |
| 15.041 | 9 | W | P | L | Y | G | N | E | G | L | | HCV Core 83 | 659 | — | 6889 | — | 0 |
| 15.045 | 9 | I | P | Q | A | V | V | D | M | V | | HCV E1 339 | 13500 | — | — | 8667 | 0 |
| 15.046 | 9 | G | P | W | L | T | P | R | C | M | | HCV E2 600 | 651 | — | — | — | 0 |
| 15.050 | 9 | T | P | A | E | T | T | V | R | L | | HCV NS3 1530 | 3484 | — | 15500 | — | 0 |
| 15.051 | 9 | A | P | P | P | S | W | D | Q | M | | HCV NS3 1598 | 1929 | — | — | — | 0 |
| 15.052 | 9 | P | P | P | S | W | D | Q | M | W | | HCV NS3 1599 | — | — | — | — | 0 |
| 15.053 | 9 | G | P | T | P | L | L | Y | R | L | | HCV NS3 1619 | 2298 | — | — | — | 0 |
| 15.055 | 9 | G | P | G | E | G | A | V | Q | W | | HCV NS4 1906 | — | — | — | — | 0 |
| 15.056 | 9 | L | P | C | E | P | E | P | D | V | | HCV NS5 2159 | — | — | — | — | 0 |
| 15.057 | 9 | E | P | E | P | D | V | A | V | L | | HCV NS5 2162 | — | — | — | — | 0 |
| 15.058 | 9 | D | P | D | L | S | D | G | S | W | | HCV NS5 2396 | — | — | 18600 | — | 0 |
| 15.059 | 9 | P | P | H | S | A | K | S | K | F | | HCV NS5 2512 | — | — | — | — | 0 |
| 15.061 | 9 | D | P | P | Q | P | E | Y | D | L | | HCV NS5 2771 | — | — | — | — | 0 |
| 15.062 | 9 | Q | P | E | Y | D | L | E | L | I | | HCV NS5 2774 | — | — | — | — | 0 |
| 16.011 | 9 | D | P | T | T | P | L | A | R | A | | HCV 2806 | — | — | — | 800 | 0 |
| 16.015 | 9 | S | P | G | E | I | N | R | V | A | | HCV 2897 | 18000 | — | — | 2811 | 0 |
| 16.018 | 9 | T | P | T | L | A | A | R | N | A | | HCV 242 | — | — | — | 5778 | 0 |
| 15.068 | 9 | D | P | D | L | A | D | Q | L | I | | HIV VIF 99 | — | — | — | — | 0 |
| 15.069 | 9 | L | P | G | R | W | K | P | K | M | | HIV POL 110 | 1440 | — | — | — | 0 |
| 15.072 | 9 | P | P | L | P | S | V | J | K | L | | HIV VIF 161 | — | — | — | — | 0 |
| 15.074 | 9 | V | P | V | K | L | K | P | G | M | | HIV POL 179 | 18000 | — | — | 1664 | 0 |
| 15.075 | 9 | K | P | G | M | D | G | P | K | V | | HIV POL 184 | — | — | — | — | 0 |
| 15.076 | 9 | T | P | Q | D | L | N | T | M | L | | HIV GAG 185 | 7200 | — | — | — | 0 |
| 15.078 | 9 | N | P | P | I | P | V | G | E | I | | HIV GAG 258 | — | — | — | — | 0 |
| 15.079 | 9 | P | P | I | P | V | G | E | I | Y | | HIV GAG 259 | — | — | — | — | 0 |
| 15.080 | 9 | G | P | K | E | P | F | R | D | Y | | HIV GAG 293 | — | — | — | — | 0 |
| 15.082 | 9 | G | P | A | A | T | L | E | E | M | | HIV GAG 343 | 3857 | — | — | — | |
| 15.085 | 9 | E | P | P | F | L | W | M | G | Y | | HIV POL 395 | — | — | — | — | 0 |
| 15.086 | 9 | D | P | E | I | V | M | H | S | F | | HIV ENV 404 | — | — | — | — | 0 |
| 15.087 | 9 | L | P | E | K | D | S | W | T | V | | HIV POL 417 | — | — | — | 885 | 0 |
| 15.089 | 9 | A | P | T | K | A | K | R | R | V | | HIV ENV 547 | 659 | — | — | — | 0 |
| 15.090 | 9 | T | P | P | L | V | K | L | W | Y | | HIV POL 590 | — | 2667 | — | 4522 | 0 |
| 15.091 | 9 | E | P | I | V | G | A | E | T | F | | HIV POL 603 | — | 2182 | 4769 | — | 0 |
| 15.092 | 9 | Q | P | D | K | S | E | S | E | L | | HIV POL 68O | 9000 | — | — | — | 0 |
| 15.093 | 9 | L | P | P | V | V | A | K | E | I | | HIV POL 759 | 9818 | — | — | — | 0 |
| 15.094 | 9 | P | P | V | V | A | K | E | I | V | | HIV POL 760 | — | — | — | — | 0 |
| 16.020 | 9 | E | P | A | A | D | G | V | G | A | | HIV NEF 38 | — | — | — | 13000 | 0 |
| 15.099 | 9 | D | P | Q | E | R | P | R | K | L | | HPV16 E6 11 | — | — | — | — | 0 |
| 15.100 | 9 | E | P | D | R | A | H | Y | N | I | | HPV16 E7 46 | — | — | 5636 | — | 0 |
| 15.101 | 9 | C | P | E | E | K | Q | R | H | L | | HPV16 E6 118 | 18000 | — | 15500 | — | 0 |
| 15.102 | 9 | G | P | K | A | T | L | Q | D | I | | HPV18 E7 3 | 13500 | — | — | — | 0 |
| 15.103 | 9 | D | P | T | R | R | P | Y | K | L | | HPV18 E6 6 | — | — | — | — | 0 |
| 15.105 | 9 | N | P | A | E | K | L | R | H | L | | HPV18 E6 113 | 509 | — | — | — | 0 |
| 16.022 | 9 | S | P | P | H | S | P | Q | G | A | | MAGE2 60 | — | — | — | 3059 | 0 |
| 15.120 | 9 | E | P | V | T | K | A | E | M | L | | MAGE2/3 128 | — | — | — | — | 0 |
| 15.121 | 9 | V | P | G | S | D | P | A | C | Y | | MAGE2/3 261 | — | — | — | — | 0 |
| 15138 | 9 | D | P | I | G | H | L | Y | I | F | | MAGE3 170 | — | 626 | 2548 | — | 0 |
| 15.157 | 9 | Y | P | P | L | H | E | W | V | L | | MAGE3 301 | 2038 | 947 | 3957 | 4522 | 0 |
| 16.023 | 9 | A | P | A | T | E | E | Q | E | A | | MAGE3 3O | — | — | — | — | 0 |
| 16.024 | 9 | D | P | P | Q | S | P | Q | G | A | | MAGE3 6O | — | — | — | 7704 | 0 |
| 15.173 | 9 | M | P | N | D | P | N | R | N | V | | CSP 293 | — | — | — | 612 | 0 |
| 15.174 | 9 | I | P | D | S | I | Q | D | S | L | | SSP2 164 | 2455 | — | — | — | 0 |
| 15.176 | 9 | G | P | F | M | K | A | V | C | V | | SSP2 228 | 2314 | — | — | — | 0 |
| 15.177 | 9 | L | P | K | R | E | P | L | D | V | | SSP2 287 | 8308 | — | — | — | 0 |
| 15.179 | 9 | P | P | N | P | P | N | P | D | I | | SSP2 364 | — | — | — | — | 0 |
| 15.180 | 9 | I | P | E | D | S | E | K | E | V | | SSP2 379 | — | — | — | — | 0 |
| 15.181 | 9 | E | P | A | P | F | D | E | T | L | | SSP2 544 | — | 2939 | 1625 | — | 0 |
| 15.183 | 9 | L | P | N | D | K | S | D | R | Y | | SSP2 419 | — | 533 | 2214 | — | 0 |
| 15.184 | 9 | L | P | S | E | N | E | R | G | Y | | LSA1 1663 | — | 2038 | — | — | 0 |
| 15.185 | 9 | K | P | I | V | Q | Y | D | N | F | | LSA1 1786 | — | 10800 | 2038 | — | 0 |
| 16.071 | 9 | D | P | Q | V | T | A | Q | D | V | | P. falciparum EXP-1 136 | — | — | — | — | 0 |
| 16.072 | 9 | E | P | L | I | D | V | H | D | L | | P. falciparum EXP-1 45 | — | — | — | — | 0 |
| 16.073 | 9 | Q | P | Q | G | D | D | N | N | L | | P. falciparum EXP-1 148 | — | — | — | — | 0 |
| 16.175 | 9 | V | P | G | A | A | T | P | Y | A | | P. falciparium TRAP 522 | — | — | — | 5778 | 0 |
| 15.217 | 10 | F | P | H | C | L | A | F | S | Y | M | HBV POL 541 | 99 | 119 | 380 | 671 | 3 |
| 15.268 | 10 | Y | P | L | A | S | L | R | S | L | F | HIV GAG 507 | 400 | 480 | 150 | 759 | 3 |
| 15.350 | 10 | T | P | Y | A | G | E | P | A | P | F | SSP2 539 | 55 | 76 | 420 | 4674 | 3 |
| 15.214 | 10 | T | P | A | R | V | T | G | G | V | F | HBV POL 365 | 75 | 294 | — | — | 2 |
| 15.225 | 10 | Y | P | C | T | V | N | F | T | I | F | HCV E2 622 | 1521 | 399 | 1257 | 315 | 2 |
| 16.185 | 10 | I | P | Q | A | V | V | D | M | V | A | HCV 339 | 7043 | 300 | — | 5.7 | 2 |
| 16.187 | 10 | L | P | C | S | F | T | T | L | P | A | HCV 674 | 422 | 24000 | — | 16 | 2 |
| 16.196 | 10 | L | P | Q | G | W | K | G | S | P | A | HIV POL 320 | 450 | — | — | 18 | 2 |
| 15.210 | 10 | L | P | L | D | K | G | I | K | P | Y | HBV POL 123 | — | 248 | — | 13 | 1 |
| 16.177 | 10 | I | P | I | P | S | S | W | A | F | A | HBV ENV 313 | 4154 | 3064 | 6643 | 23 | 1 |
| 16.180 | 10 | A | P | F | T | Q | C | G | Y | P | A | HBV POL 633 | 1895 | — | — | 77 | 1 |
| 16.181 | 10 | L | P | I | H | T | A | E | L | L | A | HBV POL 712 | 3086 | 6857 | 5813 | 32 | 1 |
| 16.182 | 10 | G | P | C | A | L | R | F | T | S | A | HBV X 67 | 60 | — | — | 3000 | 1 |
| 15.218 | 10 | L | P | R | R | G | P | R | L | G | V | HCV Core 37 | 28 | — | — | 4160 | 1 |
| 15.219 | 10 | A | P | L | G | G | A | A | R | A | L | HCV Core 142 | 9.4 | — | — | 13867 | 1 |
| 15.223 | 10 | V | P | A | S | Q | V | C | G | P | V | HCV E2 497 | 500 | — | — | 5200 | 1 |
| 15.226 | 10 | S | P | L | L | L | S | T | T | E | W | HCV E2 663 | 21600 | — | 55 | 10400 | 1 |
| 15.231 | 10 | R | P | S | G | M | F | D | S | S | V | HCV NS3 1506 | 149 | — | — | — | 1 |
| 15.234 | 10 | K | P | T | L | H | G | P | T | P | L | HCV NS3 1614 | 3.8 | — | — | — | 1 |
| 15.235 | 10 | T | P | L | L | Y | R | L | G | A | V | HCV NS3 1621 | 450 | — | — | 940 | 1 |
| 15.237 | 10 | N | P | A | I | A | S | L | M | A | F | HCV NS4 1783 | 393 | 9000 | — | — | 1 |
| 15.238 | 10 | L | P | A | I | L | S | P | G | A | L | HCV NS4 1882 | 1019 | — | — | 50 | 1 |
| 15.239 | 10 | S | P | G | A | L | V | V | G | V | V | HCV NS4 1887 | 415 | — | — | — | 1 |
| 15.247 | 10 | A | P | T | L | W | A | R | M | I | L | HCV NS5 2835 | 6.1 | — | — | — | 1 |
| 16.189 | 10 | V | P | H | P | N | I | E | E | V | A | HCV 1356 | — | — | — | 36 | 1 |
| 15.257 | 10 | I | P | V | G | E | I | Y | K | R | W | HIV GAG 261 | — | — | 175 | — | 1 |
| 15.269 | 10 | A | P | T | K | A | K | R | R | V | V | HIV ENV 547 | 44 | — | — | — | 1 |
| 15.282 | 10 | V | P | I | S | H | L | Y | I | L | V | MAGE2 170 | 2000 | — | 5580 | 100 | 1 |
| 15.283 | 10 | M | P | K | T | G | L | L | I | I | V | MAGE2 196 | 18000 | 24000 | — | 170 | 1 |
| 15.285 | 10 | H | P | R | K | L | L | M | Q | D | L | MAGE2 241 | 137 | — | — | — | 1 |
| 16.199 | 10 | F | P | D | L | E | S | E | F | Q | A | MAGE2/3 98 | — | 5760 | — | 297 | 1 |
| 15.307 | 10 | L | P | T | T | M | N | Y | P | L | W | MAGE3 71 | — | 12000 | 174 | 2950 | 1 |
| 15.311 | 10 | M | P | K | A | G | L | L | I | I | V | MAGE3 196 | 1770 | — | 14308 | 12 | 1 |
| 16.201 | 10 | D | P | I | G | H | L | Y | I | F | A | MAGE3 170 | — | — | 20667 | 359 | 1 |
| 15.208 | 10 | G | P | L | E | E | E | L | P | R | L | HBV POL 19 | — | — | — | — | 0 |
| 15.209 | 10 | I | P | W | T | H | K | V | G | N | F | HBV POL 50 | 4050 | — | — | — | 0 |
| 15.211 | 10 | P | P | N | A | P | I | L | S | T | L | HBV CORE 134 | — | — | — | — | 0 |
| 15.212 | 10 | G | P | L | L | V | L | Q | A | G | F | HBV ENV 173 | — | — | — | — | 0 |
| 15.213 | 10 | V | P | F | V | Q | W | F | V | G | L | HBV ENV 340 | 5143 | — | — | 4245 | 0 |
| 15.215 | 10 | L | P | I | F | F | C | L | W | V | Y | HBV ENV 379 | — | 917 | 16412 | — | 0 |
| 15.216 | 10 | V | P | N | L | Q | S | L | T | N | L | HBV POL 409 | 9000 | — | — | — | 0 |
| 16.178 | 10 | S | P | H | H | T | A | L | R | Q | A | HBV NUC 49 | 4500 | — | — | 3000 | 0 |
| 16.179 | 10 | T | P | P | A | Y | R | P | P | N | A | HBV NUC 128 | — | — | — | 997 | 0 |
| 15.220 | 10 | L | P | G | C | S | F | S | I | F | L | HCV Core 168 | 2512 | 8000 | 686 | 8432 | 0 |
| 15.221 | 10 | I | P | T | T | T | I | R | R | H | V | HCV E1 252 | 9818 | — | — | — | 0 |
| 15.222 | 10 | Y | P | G | H | V | S | G | H | R | M | HCV E1 308 | 1301 | 3927 | — | — | 0 |
| 15.224 | 10 | G | P | W | L | T | P | R | C | M | V | HCV E2 600 | — | — | — | — | 0 |
| 15.227 | 10 | W | P | L | L | L | L | L | L | A | L | HCV E2 793 | 1333 | — | 2620 | 2849 | 0 |
| 15.228 | 10 | T | P | C | T | C | G | S | S | D | L | HCV NS3 1120 | 10800 | — | — | — | 0 |
| 15.229 | 10 | V | P | A | A | Y | A | A | Q | G | Y | HCV NS3 1239 | — | 9600 | — | — | 0 |
| 15.230 | 10 | N | P | S | V | A | A | T | L | G | F | HCV NS3 1254 | — | — | — | — | 0 |
| 15.232 | 10 | L | P | V | C | Q | D | H | L | E | F | HCV NS3 1547 | — | 1565 | 827 | — | 0 |
| 15.233 | 10 | A | P | P | P | S | W | D | Q | M | W | HCV NS3 1598 | — | — | 15500 | — | 0 |
| 15.236 | 10 | L | P | G | N | P | A | I | A | S | L | HCV NS4 1780 | 752 | 4364 | — | 2311 | 0 |
| 15.240 | 10 | G | P | G | E | G | A | V | Q | W | M | HCV NS4 1906 | — | — | — | — | 0 |
| 15.241 | 10 | V | P | E | S | D | A | A | A | R | V | HCV NS4 1934 | — | — | — | — | 0 |
| 15.242 | 10 | E | P | D | V | A | V | L | T | S | M | HCV NS5 2164 | 3375 | 1694 | — | — | 0 |
| 15.243 | 10 | S | P | G | Q | R | V | E | F | L | V | HCV NS5 2615 | 18000 | — | — | — | 0 |
| 15.244 | 10 | P | P | G | D | P | P | Q | P | E | Y | HCV NS5 2768 | — | — | — | — | 0 |
| 15.245 | 10 | P | P | Q | P | E | Y | D | L | E | L | HCV NS5 2772 | — | — | — | — | 0 |
| 15.246 | 10 | T | P | V | N | S | W | L | G | N | I | HCV NS5 2822 | — | — | 16909 | — | 0 |
| 16.183 | 10 | D | P | S | H | I | T | A | E | T | A | HCV 2181 | 2348 | — | — | 2600 | 0 |
| 16.184 | 10 | D | P | T | T | P | L | A | R | A | A | HCV 2806 | — | — | 20667 | 800 | 0 |
| 16.186 | 10 | L | P | C | E | P | E | P | D | V | A | HCV 2159 | — | — | — | 5474 | 0 |
| 16.188 | 10 | Q | P | E | K | G | G | R | K | P | A | HCV 2567 | 4909 | — | — | 547 | 0 |
| 15.248 | 10 | E | P | V | D | P | R | L | E | P | W | HIV TAT 2 | — | — | 1603 | — | 0 |
| 15.249 | 10 | S | P | T | R | R | E | L | Q | V | W | HIV POL 37 | 2189 | — | 10941 | — | 0 |
| 15.250 | 10 | L | P | G | R | W | K | P | K | M | I | HIV POL 110 | — | — | — | — | 0 |
| 15.251 | 10 | T | P | V | N | I | I | G | R | N | L | HIV POL 152 | 18000 | — | — | — | 0 |
| 15.252 | 10 | K | P | P | L | P | S | V | J | K | L | HIV VIF 160 | 3176 | — | — | — | 0 |
| 15.253 | 10 | S | P | I | E | T | V | P | V | K | L | HIV POL 174 | 1964 | — | — | — | 0 |
| 15.254 | 10 | G | P | E | N | P | Y | N | T | P | V | HIV POL 222 | — | — | — | — | 0 |
| 15.255 | 10 | N | P | Y | N | T | P | V | F | A | I | HIV POL 225 | 1612 | — | — | 6603 | 0 |
| 15.256 | 10 | N | P | P | I | P | V | G | E | I | Y | HIV GAG 258 | — | — | — | — | 0 |
| 15.258 | 10 | V | P | L | D | K | D | F | R | K | Y | HIV POL 289 | — | 16000 | — | — | 0 |
| 15.259 | 10 | G | P | K | E | P | F | R | D | Y | V | HIV GAG 293 | — | — | — | — | 0 |
| 15.260 | 10 | E | P | F | R | D | Y | V | D | R | F | HIV GAG 296 | — | — | — | — | 0 |
| 15.261 | 10 | T | P | G | I | R | Y | Q | Y | N | V | HIV POL 310 | 13500 | — | — | — | 0 |
| 15.262 | 10 | E | P | F | R | K | Q | N | P | D | I | HIV POL 340 | — | — | — | — | 0 |
| 15.263 | 10 | G | P | A | A | T | L | E | E | M | M | HIV GAG 343 | 2700 | — | — | — | 0 |
| 15.264 | 10 | N | P | D | I | V | I | Y | Q | Y | M | HIV POL 346 | 10800 | 2057 | 7750 | 10400 | 0 |
| 15.265 | 10 | P | P | F | L | W | M | G | Y | E | L | HIV POL 396 | — | — | — | — | 0 |
| 15.266 | 10 | H | P | D | K | W | T | V | Q | P | I | HIV POL 406 | 4500 | — | — | — | 0 |
| 15.267 | 10 | E | P | T | A | P | P | E | E | S | F | HIV GAG 473 | — | — | — | — | 0 |
| 15.270 | 10 | P | P | L | V | K | L | W | Y | Q | L | HIV POL 591 | — | — | — | — | 0 |
| 15.271 | 10 | E | P | I | V | G | A | E | T | F | Y | HIV POL 603 | — | 9000 | — | — | 0 |
| 15.272 | 10 | Q | P | D | K | S | E | S | E | L | V | HIV POL 680 | — | — | — | — | 0 |
| 15.273 | 10 | L | P | P | V | V | A | K | E | I | V | HIV POL 759 | — | — | — | — | 0 |
| 15.274 | 10 | I | P | Y | N | P | Q | S | Q | G | V | HIV POL 872 | 2400 | — | — | 1841 | 0 |
| 15.275 | 10 | D | P | L | W | K | G | P | A | K | L | HIV POL 963 | — | — | — | — | 0 |
| 16.190 | 10 | G | P | G | H | K | A | R | V | L | A | HIV GAG 360 | — | — | — | 3059 | 0 |
| 16.191 | 10 | N | P | D | C | K | T | I | L | K | A | HIV GAG 332 | — | — | — | — | 0 |
| 16.192 | 10 | S | P | E | V | I | P | M | F | S | A | HIV GAG 170 | — | — | — | 5622 | 0 |
| 16.195 | 10 | I | P | A | E | T | G | Q | E | T | A | HIV POL 820 | — | — | — | 594 | 0 |
| 16.197 | 10 | P | P | v | V | A | K | E | I | V | A | HIV POL 760 | — | — | — | — | 0 |
| 15.276 | 10 | E | P | D | R | A | H | Y | N | I | V | HPV16 E7 46 | — | — | — | — | 0 |
| 15.277 | 10 | N | P | Y | A | V | C | D | K | C | L | HPV16 E6 65 | — | — | — | — | 0 |
| 15.278 | 10 | G | P | K | A | T | L | Q | D | I | V | HPV18 E7 3 | — | — | — | — | 0 |
| 15.279 | 10 | E | P | Q | N | E | I | P | V | D | L | HPV18 E7 16 | — | 16000 | — | — | 0 |
| 15.280 | 10 | E | P | Q | R | H | T | M | L | C | M | HPV18 E7 55 | 2077 | — | 2146 | — | 0 |
| 15.281 | 10 | I | P | H | A | A | C | H | K | C | I | HPV18 E6 60 | 831 | — | — | 20800 | 0 |
| 15.288 | 10 | G | P | R | A | L | I | E | T | S | Y | MAGE2 274 | 6750 | 24000 | — | — | 0 |
| 16.198 | 10 | A | P | A | T | E | E | Q | Q | T | A | MAGE2 30 | — | — | — | — | 0 |
| 15.294 | 10 | A | P | E | E | K | I | W | E | E | L | MAGE2/3 216 | — | — | — | — | 0 |
| 15.317 | 10 | D | P | K | K | L | L | T | Q | H | F | MAGE3 241 | — | — | — | — | 0 |
| 15.321 | 10 | G | P | R | A | L | V | E | T | S | Y | MAGE3 274 | — | — | — | — | 0 |
| 16.200 | 10 | A | P | A | T | E | E | Q | E | A | A | MAGE3 30 | — | — | — | — | 0 |
| 15.343 | 10 | N | P | D | P | N | A | N | P | N | V | CSP 101 | — | — | — | — | 0 |
| 15.344 | 10 | E | P | S | D | K | H | I | K | E | Y | CSP 318 | — | — | — | — | 0 |
| 15.345 | 10 | N | P | P | N | P | P | N | P | D | I | SSP2 363 | — | — | — | — | 0 |
| 15.346 | 10 | L | P | N | D | K | S | D | R | Y | I | SSP2 419 | 3857 | — | 18600 | — | 0 |
| 15.347 | 10 | I | P | Y | S | P | L | S | P | K | V | SSP2 428 | 15429 | — | — | 723 | 0 |
| 15.348 | 10 | H | P | S | D | G | K | C | N | L | Y | SSP2 206 | — | 3692 | 16909 | — | 0 |
| 15.349 | 10 | N | P | E | D | D | R | E | E | N | F | SSP2 394 | — | — | — | — | 0 |
| 15.351 | 10 | L | P | S | E | N | E | R | G | Y | Y | LSA1 1663 | — | 5538 | — | — | 0 |
| 16.218 | 10 | D | P | N | R | N | V | D | E | N | A | P. falciparum CSP 327 | — | — | — | — | 0 |
| 16.241 | 10 | E | P | L | I | D | V | H | D | L | I | P. falciparum EXP-1 45 | — | — | — | — | 0 |
| 16.242 | 10 | Q | P | Q | G | D | D | N | N | L | V | P falciparum EXP-1 148 | — | — | — | — | 0 |
| 16.243 | 10 | D | P | A | D | N | A | N | P | D | A | P falciparum EXP-1 116 | — | — | — | — | 0 |
| 16.244 | 10 | E | P | N | A | D | P | Q | V | T | A | P falciparum EXP-1 132 | — | — | — | 3302 | 0 |
| 16.307 | 10 | K | P | E | Q | K | E | D | K | S | A | P falciparum LSA1 1728 | — | — | — | — | 0 |
| 16.342 | 10 | Q | P | R | P | R | G | D | N | F | A | P falciparum TRAP 303 | 6000 | — | — | 12235 | 0 |
| 16.202 | 10 | S | P | S | C | P | L | E | R | F | A | PAP 348 | — | — | — | 2447 | 0 |
| 16.343 | 10 | E | P | A | L | G | T | T | C | Y | A | PSA 141 | — | — | — | 4522 | 0 |

TABLE 14
B7-like cross-reactive binders

| | | | | | | | | | | | | | | | | | Minimal |
|---|---|---|---|---|---|---|---|---|---|---|---|---|---|---|---|---|---|
| | | | | | | | | | | | | | B*0701 | B*3501 | B*5301 | B*54O1 | population |
| PEPTIDE | AA | P1 | P2 | P3 | P4 | P5 | P6 | P7 | P8 | P9 | P10 | Virus | (nM) | (nM) | (nM) | (nM) | coverage |
| 15.066 | 9 | F | P | V | R | P | Q | V | P | L | | HIV | 7.1 | 22 | 192 | 44 | 36.3 |
| 15.032 | 9 | I | P | I | P | S | S | W | A | F | | HBV | 60 | 7.8 | 35 | 4000 | 32.6 |
| 15.044 | 9 | L | P | G | C | S | F | S | I | F | | HCV | 61 | 113 | 122 | 8000 | 32.6 |
| 15.107 | 9 | V | P | I | S | H | L | Y | I | L | | MAGE2 | 22 | 384 | 396 | 3525 | 32.6 |
| 15.037 | 9 | F | P | H | C | L | A | F | S | Y | | HBV | 3375 | 7.5 | 18 | 400 | 25.8 |
| 15.140 | 9 | M | P | K | A | G | L | L | I | I | | MAGE3 | 320 | — | 92 | 112 | 21.6 |
| 15.134 | 9 | L | P | T | T | M | N | Y | P | L | | MAGE3 | 71 | 48 | 802 | 3152 | 27.9 |
| 16.012 | 9 | F | P | Y | L | V | A | Y | Q | A | | HCV | 18000 | 182 | 1706 | 1.2 | 20.6 |
| 16.009 | 9 | L | P | V | C | A | F | S | S | A | | HBV | 348 | 533 | — | 2.0 | 16.3 |
| 16.064 | 9 | F | P | R | I | W | L | H | J | L | | HIV | 5.4 | 10286 | 16909 | 226 | 16.3 |
| 16.032 | 9 | A | P | L | L | L | A | R | A | A | | PAP | 257 | — | — | 2.6 | 16.3 |
| 16.176 | 9 | H | P | Q | W | V | L | T | A | A | | PSA | 225 | 1532 | — | 1.1 | 16.3 |
| 15.047 | 9 | Y | P | C | T | V | N | F | T | I | | HCV | 10800 | 966 | 102 | 89 | 9.9 |
| 15.073 | 9 | F | P | I | S | P | I | E | T | V | | HIV | 3484 | 1051 | 251 | 9.8 | 9.9 |
| 15.217 | 10 | F | P | H | C | L | A | F | S | Y | M | HBV | 99 | 119 | 380 | 671 | 32.6 |
| 15.268 | 10 | Y | P | L | A | S | L | R | S | L | F | HIV | 400 | 480 | 150 | 759 | 32.6 |
| 15.350 | 10 | T | P | Y | A | G | E | P | A | P | F | P fal | 55 | 76 | 420 | 4674 | 32.6 |
| 15.214 | 10 | T | P | A | R | V | T | G | G | V | F | HBV | 75 | 294 | — | — | 27.9 |
| 15.225 | 10 | Y | P | C | T | V | N | F | T | I | F | HCV | 1521 | 399 | 1257 | 315 | 20.6 |
| 16.185 | 10 | I | P | Q | A | V | V | D | M | V | A | HCV | 7043 | 300 | — | 5.7 | 20.6 |
| 16.187 | 10 | L | P | C | S | F | T | T | L | P | A | HCV | 422 | 24000 | — | 16 | 16.3 |
| 16.196 | 10 | L | P | Q | G | W | K | G | S | P | A | HIV | 450 | — | — | 18 | 16.3 |

TABLE 15
Improved prediction of B7-like supermotif
cross-reactive peptides

| | No. of Cross- | Fraction of |
|---|---|---|
| | reactive Peptides | Cross-reactive |
| | Predicted | Peptides Predicted |
| Selection Criteria | ≧2 alleles bound | ≧2 alleles bound |
| none observed | 14/24 (11%) | 14/14 (100%) |
| no negative residues present | 13/54 (24%) | 13/14 (93%) |
| no negative residues present | 12/25 (48%) | 12/14 (86%) |
| at least one preferred residue | | |
| present | | |

What is claimed is:

1. A composition comprising an immunogenic peptide having a supermotif which allows the immunogenic peptide to bind more than one HLA molecule, the immunogenic peptide having between about 9 and about 10 residues;
a first conserv residue at the second position from the N-terminus being P; and
a second conse ed residue at the C-terminal position being selected from the group consisting of M, I, d an aromatic residue.

2. e composition of claim 1, wherein the second conserved residue is selected from the gr p consisting of I and M.

3. Th composition of claim 1, wherein the second conserved residue is an aromatic residue selted from the group consisting of F, W, and Y.

4. A mposition of claim 1, wherein the N-terminal residue is selected from the group c nsisting of Y, F and W.

5. The mposition of claim 1, wherein the residue at the fourth position from the N-termins is selected from the group consisting of S, T and C.

6. The c mposition of claim 1, wherein the residue at the eighth position from the N-terminu is selected from the group consisting of A and P.

7. The position of claim 1, wherein the immunogenic peptide consists of 9 residues.

8. The co position of claim 1, wherein the immunogenic peptide is derived from a parasitic antig 9. The compsition of claim 8, wherein the parasitic antigen is from Plasmodium falciparum.
23; The composition of claim 9, wherein the immunogenic peptide comprises the sequence TPYAGENPAPF.

10. The composition of claim 1, wherein the immunogenic peptide is derived from a viral antigen.

11. The composition of claim 10, wherein the viral antigen is from HIV, HBV, HCV, or HPV.

12. The composition of claim 11, wherein the immunogenic peptide comprises the amino acid sequence PRVRPQVPL or the sequence YPLASLRSLF.

13. The composition of claim 11, wherein the immunogenic peptide comprises an amino acid sequence selected from the group consisting of IPIPSSWAF, FPHCLAFSYM and TPARVITGGVF.

14. The conposition of claim 11, wherein the immunogenic peptide comprises the sequence LPGCSFSIF.

15. The composition of claim 1, wherein the immunogenic peptide is derived from an antigen associated with cancer.

16. The composition of claim 15, wherein the antigen is MAGE-1, MAGE-2, MAGE-3, or PSA. Aderived from an antigen as ociated with cancer.

17. The composition of claim 16, wherein the immunogenic peptide comprises the sequence VPISHLYIL.

18. The composition of claims 16, wherein the immunogenic peptide comprises the sequence LPTTMNYPL.

19. A pharmaceutical composition comprising a pgarnacetucally acceptable carrier and the immunogenic peptide of claim 1. Ao<ethod for inducing a CTL response in a patient, the method <Comprising adminito the patient a therapeutically effective dose of the A mmunogenic peptide of cam 1. /,. The m of claimn, wherein the immunogenic peptide induces a CTL response against cellxpressing an antigen associated with cancer. c , The composition of claim 1, wherein the immunogenic peptide is expressed by an attenuated recombinant viraorb al host.

7 The composition o [e in the viral host is vaccinia.
